SEQUENCE LISTING (613 sequences)

SEQ ID NO: 1 - protein, 225 aa, Homo sapiens
IVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 2 - protein, 4 aa, Artificial sequence
  peptide fragment amino acid residues 7-10 of hHBP
ARPR

SEQ ID NO: 3 - protein, 4 aa, Artificial sequence
  peptide fragment amino acid residues 8-11 of hHBP
RPRQ

SEQ ID NO: 4 - protein, 4 aa, Artificial sequence
  peptide fragment amino acid residues 9-12 of hHBP
PRQF

SEQ ID NO: 5 - protein, 4 aa, Artificial sequence
  peptide fragment amino acid residues 10-13 of hHBP
RQFQ

SEQ ID NO: 6 - protein, 4 aa, Artificial sequence
  peptide fragment amino acid residues 11-14 of hHBP
QFQF

SEQ ID NO: 7 - protein, 4 aa, Artificial sequence
  peptide fragment amino acid residues 12-15 of hHBP
FQFL

SEQ ID NO: 8 - protein, 4 aa, Artificial sequence
  peptide fragment amino acid residues 13-16 of hHBP
QFLA

SEQ ID NO: 9 - protein, 4 aa, Artificial sequence
  peptide fragment amino acid residues 14-17 of hHBP
FLAS

SEQ ID NO: 10 - protein, 4 aa, Artificial sequence
  peptide fragment amino acid residues 15-18 of hHBP
LASI

SEQ ID NO: 11 - protein, 4 aa, Artificial sequence
  peptide fragment amino acid residues 16-19 of hHBP
ASIQ

SEQ ID NO: 12 - protein, 4 aa, Artificial sequence
  peptide fragment amino acid residues 17-20 of hHBP
SIQN

SEQ ID NO: 13 - protein, 4 aa, Artificial sequence
  peptide fragment amino acid residues 18-21 of hHBP
IQNQ

SEQ ID NO: 14 - protein, 4 aa, Artificial sequence
  peptide fragment amino acid residues 19-22 of hHBP
QNQG

SEQ ID NO: 15 - protein, 4 aa, Artificial sequence
  peptide fragment amino acid residues 20-23 of hHBP
NQGR

SEQ ID NO: 16 - protein, 4 aa, Artificial sequence
  peptide fragment amino acid residues 21-24 of hHBP
QGRH

SEQ ID NO: 17 - protein, 4 aa, Artificial sequence
  peptide fragment amino acid residues 22-25 of hHBP
GRHF

SEQ ID NO: 18 - protein, 4 aa, Artificial sequence
  peptide fragment amino acid residues 23-26 of hHBP
RHFC

SEQ ID NO: 19 - protein, 4 aa, Artificial sequence
  peptide fragment amino acid residues 24-27 of hHBP
HFCG

SEQ ID NO: 20 - protein, 4 aa, Artificial sequence
  peptide fragment amino acid residues 25-28 of hHBP
FCGG

SEQ ID NO: 21 - protein, 4 aa, Artificial sequence
  peptide fragment amino acid residues 26-29 of hHBP
CGGA

SEQ ID NO: 22 - protein, 4 aa, Artificial sequence
  peptide fragment amino acid residues 27-30 of hHBP
GGAL

SEQ ID NO: 23 - protein, 4 aa, Artificial sequence
  peptide fragment amino acid residues 28-31 of hHBP
GALI

SEQ ID NO: 24 - protein, 4 aa, Artificial sequence
  peptide fragment amino acid residues 29-32 of hHBP
ALIH

SEQ ID NO: 25 - protein, 4 aa, Artificial sequence
  peptide fragment amino acid residues 30-33 of hHBP
LIHA

SEQ ID NO: 26 - protein, 4 aa, Artificial sequence
  peptide fragment amino acid residues 31-34 of hHBP
IHAR

SEQ ID NO: 27 - protein, 4 aa, Artificial sequence
  peptide fragment amino acid residues 32-35 of hHBP
HARF

SEQ ID NO: 28 - protein, 4 aa, Artificial sequence
  Peptide fragment amino acid residues 33-36 of hHBP
ARFV

SEQ ID NO: 29 - protein, 4 aa, Artificial sequence
  Peptide fragment amino acid residues 34-37 of hHBP
RFVM

SEQ ID NO: 30 - protein, 4 aa, Artificial sequence
  Peptide fragment amino acid residues 35-38 of hHBP
FVMT

SEQ ID NO: 31 - protein, 4 aa, Artificial sequence
  Peptide fragment amino acid residues 36-39 of hHBP
VMTA

SEQ ID NO: 32 - protein, 4 aa, Artificial sequence
  Peptide fragment amino acid residues 37-40 of hHBP
MTAA

SEQ ID NO: 33 - protein, 4 aa, Artificial sequence
  Peptide fragment amino acid residues 38-41 of hHBP
TAAS

SEQ ID NO: 34 - protein, 4 aa, Artificial sequence
  Peptide fragment amino acid residues 39-42 of hHBP
AASC

SEQ ID NO: 35 - protein, 4 aa, Artificial sequence
  Peptide fragment amino acid residues 40-43 of hHBP
ASCF

SEQ ID NO: 36 - protein, 4 aa, Artificial sequence
  Peptide fragment amino acid residues 41-44 of hHBP
SCFQ

SEQ ID NO: 37 - protein, 4 aa, Artificial sequence
  Peptide fragment amino acid residues 42-45 of hHBP
CFQS

SEQ ID NO: 38 - protein, 4 aa, Artificial sequence
  Peptide fragment amino acid residues 43-46 of hHBP
FQSQ

SEQ ID NO: 39 - protein, 4 aa, Artificial sequence
  Peptide fragment amino acid residues44-47 of hHBP
QSQN

SEQ ID NO: 40 - protein, 4 aa, Artificial sequence
  Peptide fragment amino acid residues 45-48 of hHBP
SQNP

SEQ ID NO: 41 - protein, 4 aa, Artificial sequence
  Peptide fragment amino acid residues46-49 of hHBP
QNPG

SEQ ID NO: 42 - protein, 4 aa, Artificial sequence
  Peptide fragment amino acid residues 47-50 of hHBP
NPGV

SEQ ID NO: 43 - protein, 4 aa, Artificial sequence
  Peptide fragment amino acid residues 48-51 of hHBP
PGVS

SEQ ID NO: 44 - protein, 4 aa, Artificial sequence
  Peptide fragment amino acid residues 49-52 of hHBP
GVST

SEQ ID NO: 45 - protein, 4 aa, Artificial sequence
  Peptide fragment amino acid residues 50-53 of hHBP
VSTV

SEQ ID NO: 46 - protein, 4 aa, Artificial sequence
  Peptide fragment amino acid residues 51-54 of hHBP
STVV

SEQ ID NO: 47 - protein, 4 aa, Artificial sequence
  Peptide fragment amino acid residues 52-55 of hHBP
TVVL

SEQ ID NO: 48 - protein, 4 aa, Artificial sequence
  Peptide fragment amino acid residues 53-56 of hHBP
VVLG

SEQ ID NO: 49 - protein, 4 aa, Artificial sequence
  Peptide fragment amino acid residues 54-57 of hHBP
VLGA

SEQ ID NO: 50 - protein, 4 aa, Artificial sequence
  Peptide fragment amino acid residues 55-58 of hHBP
LGAY

SEQ ID NO: 51 - protein, 4 aa, Artificial sequence
  Peptide fragment amino acid residues 56-59 of hHBP
GAYD

SEQ ID NO: 52 - protein, 4 aa, Artificial sequence
  Peptide fragment amino acid residues 57-60 of hHBP
AYDL

SEQ ID NO: 53 - protein, 4 aa, Artificial sequence
  Peptide fragment amino acid residues 58-61 of hHBP
YDLR

SEQ ID NO: 54 - protein, 4 aa, Artificial sequence
  Peptide fragment amino acid residues 59-62 of hHBP
DLRR

SEQ ID NO: 55 - protein, 4 aa, Artificial sequence
  Peptide fragment amino acid residues 60-63 of hHBP
LRRR

SEQ ID NO: 56 - protein, 4 aa, Artificial sequence
  Peptide fragment amino acid residues 61-62 of hHBP
RRRE

SEQ ID NO: 57 - protein, 4 aa, Artificial sequence
  Peptide fragment amino acid residues 62-65 of hHBP
RRER

SEQ ID NO: 58 - protein, 4 aa, Artificial sequence
  Peptide fragment amino acid residues 63-66 of hHBP
RERQ

SEQ ID NO: 59 - protein, 4 aa, Artificial sequence
  Peptide fragment amino acid residues 64-67 of hHBP
ERQS

SEQ ID NO: 60 - protein, 4 aa, Artificial sequence
  Peptide fragment amino acid residues 65-68 of hHBP
RQSR

SEQ ID NO: 61 - protein, 4 aa, Artificial sequence
  Peptide fragment amino acid residues 66-69 of hHBP
QSRQ

SEQ ID NO: 62 - protein, 4 aa, Artificial sequence
  Peptide fragment amino acid residues 67-70 of hHBP
SRQT

SEQ ID NO: 63 - protein, 4 aa, Artificial sequence
  Peptide fragment amino acid residues 68-71 of hHBP
RQTF

SEQ ID NO: 64 - protein, 4 aa, Artificial sequence
  Peptide fragment amino acid residues 69-72 of hHBP
QTFS

SEQ ID NO: 65 - protein, 4 aa, Artificial sequence
  Peptide fragment amino acid residues 70-73 of hHBP
TFSI

SEQ ID NO: 66 - protein, 4 aa, Artificial sequence
  Peptide fragment amino acid residues 71-74 of hHBP
FSIS

SEQ ID NO: 67 - protein, 4 aa, Artificial sequence
  Peptide fragment amino acid residues 72-75 of hHBP
SISS

SEQ ID NO: 68 - protein, 4 aa, Artificial sequence
  Peptide fragment amino acid residues 73-76 of hHBP
ISSM

SEQ ID NO: 69 - protein, 4 aa, Artificial sequence
  Peptide fragment amino acid residues 74-77 of hHBP
SSMS

SEQ ID NO: 70 - protein, 4 aa, Artificial sequence
  Peptide fragment amino acid residues 75-78 of hHBP
SMSE

SEQ ID NO: 71 - protein, 4 aa, Artificial sequence
  Peptide fragment amino acid residues 76-79 of hHBP
MSEN

SEQ ID NO: 72 - protein, 4 aa, Artificial sequence
  Peptide fragment amino acid residues 77-80 of hHBP
SENG

SEQ ID NO: 73 - protein, 4 aa, Artificial sequence
  Peptide fragment amino acid residues 78-81 of hHBP
ENGY

SEQ ID NO: 74 - protein, 4 aa, Artificial sequence
  Peptide fragment amino acid residues 79-82 of hHBP
NGYD

SEQ ID NO: 75 - protein, 4 aa, Artificial sequence
  Peptide fragment amino acid residues 80-83 of hHBP
GYDP

SEQ ID NO: 76 - protein, 4 aa, Artificial sequence
  Peptide fragment amino acid residues 81-84 of hHBP
YDPQ

SEQ ID NO: 77 - protein, 4 aa, Artificial sequence
  Peptide fragment amino acid residues 82-85 of hHBP
DPQQ

SEQ ID NO: 78 - protein, 4 aa, Artificial sequence
  Peptide fragment amino acid residues 83-86 of hHBP
PQQN

SEQ ID NO: 79 - protein, 4 aa, Artificial sequence
  Peptide fragment amino acid residues 84-87 of hHBP
QQNL

SEQ ID NO: 80 - protein, 4 aa, Artificial sequence
  Peptide fragment amino acid residues 85-88 of hHBP
QNLN

SEQ ID NO: 81 - protein, 4 aa, Artificial sequence
  Peptide fragment amino acid residues 86-89 of hHBP
NLND

SEQ ID NO: 82 - protein, 4 aa, Artificial sequence
  Peptide fragment amino acid residues 87-90 of hHBP
LNDL

SEQ ID NO: 83 - protein, 4 aa, Artificial sequence
  Peptide fragment amino acid residues 88-91 of hHBP
NDLM

SEQ ID NO: 84 - protein, 4 aa, Artificial sequence
  Peptide fragment amino acid residues 89-92 of hHBP
DLML

SEQ ID NO: 85 - protein, 4 aa, Artificial sequence
  Peptide fragment amino acid residues 90-93 of hHBP
LMLL

SEQ ID NO: 86 - protein, 4 aa, Artificial sequence
  Peptide fragment amino acid residues 91-94 of hHBP
MLLQ

SEQ ID NO: 87 - protein, 4 aa, Artificial sequence
  Peptide fragment amino acid residues 92-95 of hHBP
LLQL

SEQ ID NO: 88 - protein, 4 aa, Artificial sequence
  Peptide fragment amnino acid residues 93-96 of hHBP
LQLD

SEQ ID NO: 89 - protein, 4 aa, Artificial sequence
  Peptide fragment amnino acid residues 94-97 of hHBP
QLDR

SEQ ID NO: 90 - protein, 4 aa, Artificial sequence
  Peptide fragment amnino acid residues 95-98 of hHBP
LDRE

SEQ ID NO: 91 - protein, 4 aa, Artificial sequence
  Peptide fragment amnino acid residues 96-99 of hHBP
DREA

SEQ ID NO: 92 - protein, 4 aa, Artificial sequence
  Peptide fragment amnino acid residues 97-100 of hHBP
REAN

SEQ ID NO: 93 - protein, 4 aa, Artificial sequence
  Peptide fragment amino acid residues 98-101 of hHBP
EANL

SEQ ID NO: 94 - protein, 4 aa, Artificial sequence
  Peptide fragment amino acid residues 100-103 of hHBP
ANLT

SEQ ID NO: 95 - protein, 4 aa, Artificial sequence
  Peptide fragment amino acid residues 101-104 of hHBP
NLTS

SEQ ID NO: 96 - protein, 4 aa, Artificial sequence
  Peptide fragment amino acid residues 101-104 of hHBP
LTSS

SEQ ID NO: 97 - protein, 4 aa, Artificial sequence
  Peptide fragment amino acid residues 102-105 of hHBP
TSSV

SEQ ID NO: 98 - protein, 4 aa, Artificial sequence
  Peptide fragment amino acid residues 103-106 of hHBP
SSVT

SEQ ID NO: 99 - protein, 4 aa, Artificial sequence
  Peptide fragment amino acid residues 104-107 of hHBP
SVTI

SEQ ID NO: 100 - protein, 4 aa, Artificial sequence
  Peptide fragment amino acid residues 105-108 of hHBP
VTIL

SEQ ID NO: 101 - protein, 4 aa, Artificial sequence
  Peptide fragment amino acid residues 106-109 of hHBP
TILP

SEQ ID NO: 102 - protein, 4 aa, Artificial sequence
  Peptide fragment amino acid residues 107-110 of hHBP
ILPL

SEQ ID NO: 103 - protein, 4 aa, Artificial sequence
  Peptide fragment amino acid residues 108-111 of hHBP
LPLP

SEQ ID NO: 104 - protein, 4 aa, Artificial sequence
  Peptide fragment amino acid residues 109-112 of hHBP
PLPL

SEQ ID NO: 105 - protein, 4 aa, Artificial sequence
  Peptide fragment amino acid residues 110-113 of hHBP
LPLQ

SEQ ID NO: 106 - protein, 4 aa, Artificial sequence
  Peptide fragment amino acid residues 111-114 of hHBP
PLQN

SEQ ID NO: 107 - protein, 4 aa, Artificial sequence
  Peptide fragment amino acid residues 112-115 of hHBP
LQNA

SEQ ID NO: 108 - protein, 4 aa, Artificial sequence
  Peptide fragment amino acid residues 113-116 of hHBP
QNAT

SEQ ID NO: 109 - protein, 4 aa, Artificial sequence
  Peptide fragment amino acid residues 114-117 of hHBP
NATV

SEQ ID NO: 110 - protein, 4 aa, Artificial sequence
  Peptide fragment amino acid residues 115-118 of hHBP
ATVE

SEQ ID NO: 111 - protein, 4 aa, Artificial sequence
  Peptide fragment amino acid residues 116-119 of hHBP
TVEA

SEQ ID NO: 112 - protein, 4 aa, Artificial sequence
  Peptide fragment amino acid residues 117-120 of hHBP
VEAG

SEQ ID NO: 113 - protein, 4 aa, Artificial sequence
  Peptide fragment amino acid residues 118-121 of hHBP
EAGT

SEQ ID NO: 114 - protein, 4 aa, Artificial sequence
  Peptide fragment amino acid residues 119-122 of hHBP
AGTR

SEQ ID NO: 115 - protein, 4 aa, Artificial sequence
  Peptide fragment amino acid residues 120-123 of hHBP
GTRC

SEQ ID NO: 116 - protein, 4 aa, Artificial sequence
  Peptide fragment amino acid residues 121-124 of hHBP
TRCQ

SEQ ID NO: 117 - protein, 4 aa, Artificial sequence
  Peptide fragment amino acid residues 122-125 of hHBP
RCQV

SEQ ID NO: 118 - protein, 4 aa, Artificial sequence
  Peptide fragment amino acid residues 123-126 of hHBP
CQVA

SEQ ID NO: 119 - protein, 4 aa, Artificial sequence
  Peptide fragment amino acid residues 124-127 of hHBP
QVAG

SEQ ID NO: 120 - protein, 4 aa, Artificial sequence
  Peptide fragment amino acid residues 125-128 of hHBP
VAGW

SEQ ID NO: 121 - protein, 4 aa, Artificial sequence
  Peptide fragment amino acid residues 125-128 of hHBP
AGWG

SEQ ID NO: 122 - protein, 4 aa, Artificial sequence
  Peptide fragment amino acid residues 127-130 of hHBP
GWGS

SEQ ID NO: 123 - protein, 4 aa, Artificial sequence
  Peptide fragment amino acid residues128-131 of hHBP
WGSQ

SEQ ID NO: 124 - protein, 4 aa, Artificial sequence
  Peptide fragment amino acid residues 129-132 of hHBP
GSQR

SEQ ID NO: 125 - protein, 4 aa, Artificial sequence
  Peptide fragment amino acid residues 130-133 of hHBP
SQRS

SEQ ID NO: 126 - protein, 4 aa, Artificial sequence
  Peptide fragment amino acid residues 131-134 of hHBP
QRSG

SEQ ID NO: 127 - protein, 4 aa, Artificial sequence
  Peptide fragment amino acid residues 132-135 of hHBP
RSGG

SEQ ID NO: 128 - protein, 4 aa, Artificial sequence
  Peptide fragment amino acid residues 133-136 of hHBP
SGGR

SEQ ID NO: 129 - protein, 4 aa, Artificial sequence
  Peptide fragment amino acid residues 134-137 of hHBP
GGRL

SEQ ID NO: 130 - protein, 4 aa, Artificial sequence
  Peptide fragment amino acid residues 135-138 of hHBP
GRLS

SEQ ID NO: 131 - protein, 4 aa, Artificial sequence
  Peptide fragment amino acid residues 136-139 of hHBP
RLSR

SEQ ID NO: 132 - protein, 4 aa, Artificial sequence
  Peptide fragment amino acid residues 137-140 of hHBP
LSRF

SEQ ID NO: 133 - protein, 4 aa, Artificial sequence
  Peptide fragment amino acid residues 138-141 of hHBP
SRFP

SEQ ID NO: 134 - protein, 4 aa, Artificial sequence
  Peptide fragment amino acid residues 139-142 of hHBP
RFPR

SEQ ID NO: 135 - protein, 4 aa, Artificial sequence
  Peptide fragment amino acid residues 140-143 of hHBP
FPRF

SEQ ID NO: 136 - protein, 4 aa, Artificial sequence
  Peptide fragment amino acid residues 141-144 of hHBP
PRFV

SEQ ID NO: 137 - protein, 4 aa, Artificial sequence
  Peptide fragment amino acid residues 142-145 of hHBP
RFVN

SEQ ID NO: 138 - protein, 4 aa, Artificial sequence
  Peptide fragment amino acid residues 143-146 of hHBP
FVNV

SEQ ID NO: 139 - protein, 4 aa, Artificial sequence
  Peptide fragment amino acid residues 144-147 of hHBP
VNVT

SEQ ID NO: 140 - protein, 4 aa, Artificial sequence
  Peptide fragment amino acid residues 145-148 of hHBP
NVTV

SEQ ID NO: 141 - protein, 4 aa, Artificial sequence
  Peptide fragment amino acid residues 146-149 of hHBP
VTVT

SEQ ID NO: 142 - protein, 4 aa, Artificial sequence
  Peptide fragment amino acid residues 147-150 of hHBP
TVTP

SEQ ID NO: 143 - protein, 4 aa, Artificial sequence
  Peptide fragment amino acid residues 148-151 of hHBP
VTPE

SEQ ID NO: 144 - protein, 4 aa, Artificial sequence
  Peptide fragment amino acid residues 149-152 of hHBP
TPED

SEQ ID NO: 145 - protein, 4 aa, Artificial sequence
  Peptide fragment amino acid residues 150-153 of hHBP
PEDQ

SEQ ID NO: 146 - protein, 4 aa, Artificial sequence
  Peptide fragment amino acid residues 151-154 of hHBP
EDQC

SEQ ID NO: 147 - protein, 4 aa, Artificial sequence
  Peptide fragment amino acid residues 152-155 of hHBP
DQCR

SEQ ID NO: 148 - protein, 4 aa, Artificial sequence
  Peptide fragment amino acid residues 153-156 of hHBP
QCRP

SEQ ID NO: 149 - protein, 4 aa, Artificial sequence
  Peptide fragment amino acid residues 154-157 of hHBP
CRPN

SEQ ID NO: 150 - protein, 4 aa, Artificial sequence
  Peptide fragment amino acid residues 155-158 of hHBP
RPNN

SEQ ID NO: 151 - protein, 4 aa, Artificial sequence
  Peptide fragment amino acid residues 156-159 of hHBP
PNNV

SEQ ID NO: 152 - protein, 4 aa, Artificial sequence
  Peptide fragment amino acid residues 157-160 of hHBP
NNVC

SEQ ID NO: 153 - protein, 4 aa, Artificial sequence
  Peptide fragment amino acid residues 158-161 of hHBP
NVCT

SEQ ID NO: 154 - protein, 4 aa, Artificial sequence
  Peptide fragment amino acid residues 159-162 of hHBP
VCTG

SEQ ID NO: 155 - protein, 4 aa, Artificial sequence
  Peptide fragment amino acid residues 160-163 of hHBP
CTGV

SEQ ID NO: 156 - protein, 4 aa, Artificial sequence
  Peptide fragment amino acid residues 161-164 of hHBP
TGVL

SEQ ID NO: 157 - protein, 4 aa, Artificial sequence
  Peptide fragment amino acid residues 162-165 of hHBP
GVLT

SEQ ID NO: 158 - protein, 4 aa, Artificial sequence
  Peptide fragment amino acid residues 163-166 of hHBP
VLTR

SEQ ID NO: 159 - protein, 4 aa, Artificial sequence
  Peptide fragment amino acid residues 164-167 of hHBP
LTRR

SEQ ID NO: 160 - protein, 4 aa, Artificial sequence
  Peptide fragment amino acid residues 165-168 of hHBP
TRRG

SEQ ID NO: 161 - protein, 4 aa, Artificial sequence
  Peptide fragment amino acid residues 166-169 of hHBP
RRGG

SEQ ID NO: 162 - protein, 4 aa, Artificial sequence
  Peptide fragment amino acid residues 167-170 of hHBP
RGGI

SEQ ID NO: 163 - protein, 4 aa, Artificial sequence
  Peptide fragment amino acid residues 168-171 of hHBP
GGIC

SEQ ID NO: 164 - protein, 4 aa, Artificial sequence
  Peptide fragment amino acid residues 169-172 of hHBP
GICN

SEQ ID NO: 165 - protein, 4 aa, Artificial sequence
  Peptide fragment amino acid residues 170-173 of hHBP
ICNG

SEQ ID NO: 166 - protein, 4 aa, Artificial sequence
  Peptide fragment amino acid residues 171-174 of hHBP
CNGD

SEQ ID NO: 167 - protein, 4 aa, Artificial sequence
  Peptide fragment amino acid residues 172-175 of hHBP
NGDG

SEQ ID NO: 168 - protein, 4 aa, Artificial sequence
  Peptide fragment amino acid residues 173-176 of hHBP
GDGG

SEQ ID NO: 169 - protein, 4 aa, Artificial sequence
  Peptide fragment amino acid residues 174-177 of hHBP
DGGT

SEQ ID NO: 170 - protein, 4 aa, Artificial sequence
  Peptide fragment amino acid residues 175-178 of hHBP
GGTP

SEQ ID NO: 171 - protein, 4 aa, Artificial sequence
  Peptide fragment amino acid residues 176-179 of hHBP
GTPL

SEQ ID NO: 172 - protein, 4 aa, Artificial sequence
  Peptide fragment amino acid residues 177-180 of hHBP
TPLV

SEQ ID NO: 173 - protein, 4 aa, Artificial sequence
  Peptide fragment amino acid residues 178-181 of hHBP
PLVC

SEQ ID NO: 174 - protein, 4 aa, Artificial sequence
  Peptide fragment amino acid residues 179-181 of hHBP
LVCE

SEQ ID NO: 175 - protein, 4 aa, Artificial sequence
  Peptide fragment amino acid residues 180-183 of hHBP
VCEG

SEQ ID NO: 176 - protein, 4 aa, Artificial sequence
  Peptide fragment amino acid residues 181-184 of hHBP
CEGL

SEQ ID NO: 177 - protein, 4 aa, Artificial sequence
  Peptide fragment amino acid residues 182-185 of hHBP
EGLA

SEQ ID NO: 178 - protein, 4 aa, Artificial sequence
  Peptide fragment amino acid residues 183-186 of hHBP
GLAH

SEQ ID NO: 179 - protein, 4 aa, Artificial sequence
  Peptide fragment amino acid residues 184-187 of hHBP
LAHG

SEQ ID NO: 180 - protein, 4 aa, Artificial sequence
  Peptide fragment amino acid residues 185-188 of hHBP
AHGV

SEQ ID NO: 181 - protein, 4 aa, Artificial sequence
  Peptide fragment amino acid residues 186-189 of hHBP
HGVA

SEQ ID NO: 182 - protein, 4 aa, Artificial sequence
  Peptide fragment amino acid residues 187-190 of hHBP
GVAS

SEQ ID NO: 183 - protein, 4 aa, Artificial sequence
  Peptide fragment amino acid residues 188-191 of hHBP
VASF

SEQ ID NO: 184 - protein, 4 aa, Artificial sequence
  Peptide fragment amino acid residues 189-192 of hHBP
ASFS

SEQ ID NO: 185 - protein, 4 aa, Artificial sequence
  Peptide fragment amino acid residues 190-193 of hHBP
SFSL

SEQ ID NO: 186 - protein, 4 aa, Artificial sequence
  Peptide fragment amino acid residues 191-194 of hHBP
FSLG

SEQ ID NO: 187 - protein, 4 aa, Artificial sequence
  Peptide fragment amino acid residues 192-195 of hHBP
SLGP

SEQ ID NO: 188 - protein, 4 aa, Artificial sequence
  Peptide fragment amino acid residues 193-196 of hHBP
LGPC

SEQ ID NO: 189 - protein, 4 aa, Artificial sequence
  Peptide fragment amino acid residues 194-197 of hHBP
GPCG

SEQ ID NO: 190 - protein, 4 aa, Artificial sequence
  Peptide fragment amino acid residues 195-198 of hHBP
PCGR

SEQ ID NO: 191 - protein, 4 aa, Artificial sequence
  Peptide fragment amino acid residues 196-199 of hHBP
CGRG

SEQ ID NO: 192 - protein, 4 aa, Artificial sequence
  Peptide fragment amino acid residues 197-200 of hHBP
GRGP

SEQ ID NO: 193 - protein, 4 aa, Artificial sequence
  Peptide fragment amino acid residues 198-201 of hHBP
RGPD

SEQ ID NO: 194 - protein, 4 aa, Artificial sequence
  Peptide fragment amino acid residues 199-202 of hHBP
GPDF

SEQ ID NO: 195 - protein, 4 aa, Artificial sequence
  Peptide fragment amino acid residues 200-203 of hHBP
PDFF

SEQ ID NO: 196 - protein, 4 aa, Artificial sequence
  Peptide fragment amino acid residues 201-204 of hHBP
DFFT

SEQ ID NO: 197 - protein, 4 aa, Artificial sequence
  Peptide fragment amino acid residues 202-205 of hHBP
FFTR

SEQ ID NO: 198 - protein, 4 aa, Artificial sequence
  Peptide fragment amino acid residues 203-206 of hHBP
FTRV

SEQ ID NO: 199 - protein, 4 aa, Artificial sequence
  Peptide fragment amino acid residues 204-207 of hHBP
TRVA

SEQ ID NO: 200 - protein, 4 aa, Artificial sequence
  Peptide fragment amino acid residues 205-208 of hHBP
RVAL

SEQ ID NO: 201 - protein, 4 aa, Artificial sequence
  Peptide fragment amino acid residues 206-209 of hHBP
VALF

SEQ ID NO: 202 - protein, 4 aa, Artificial sequence
  Peptide fragment amino acid residues 207-210 of hHBP
ALFR

SEQ ID NO: 203 - protein, 4 aa, Artificial sequence
  Peptide fragment amino acid residues 208-211 of hHBP
LFRD

SEQ ID NO: 204 - protein, 4 aa, Artificial sequence
  Peptide fragment amino acid residues 209-212 of hHBP
FRDW

SEQ ID NO: 205 - protein, 4 aa, Artificial sequence
  Peptide fragment amino acid residues 210-213 of hHBP
RDWI

SEQ ID NO: 206 - protein, 4 aa, Artificial sequence
  Peptide fragment amino acid residues 211-214 of hHBP
DWID

SEQ ID NO: 207 - protein, 4 aa, Artificial sequence
  Peptide fragment amino acid residues 212-215 of hHBP
WIDG

SEQ ID NO: 208 - protein, 4 aa, Artificial sequence
  Peptide fragment amino acid residues 213-216 of hHBP
IDGV

SEQ ID NO: 209 - protein, 4 aa, Artificial sequence
  Peptide fragment amino acid residues 214-217 of hHBP
DGVL

SEQ ID NO: 210 - protein, 4 aa, Artificial sequence
  Peptide fragment amino acid residues 215-218 of hHBP
GVLN

SEQ ID NO: 211 - protein, 4 aa, Artificial sequence
  Peptide fragment amino acid residues 216-219 of hHBP
VLNN

SEQ ID NO: 212 - protein, 4 aa, Artificial sequence
  Peptide fragment amino acid residues 217-220 of hHBP
LNNP

SEQ ID NO: 213 - protein, 4 aa, Artificial sequence
  Peptide fragment amino acid residues 218-221 of hHBP
NNPG

SEQ ID NO: 214 - protein, 4 aa, Artificial sequence
  Peptide fragment amino acid residues 219-222 of hHBP
NPGP

SEQ ID NO: 215 - protein, 4 aa, Artificial sequence
  Peptide fragment amino acid residues 220-223 of hHBP
PGPG

SEQ ID NO: 216 - protein, 4 aa, Artificial sequence
  Peptide fragment amino acid residues 221-224 of hHBP
GPGP

SEQ ID NO: 217 - protein, 4 aa, Artificial sequence
  Peptide fragment amino acid residues 222-225 of hHBP
PGPA

SEQ ID NO: 218 - protein, 4 aa, Artificial sequence
  Peptide fragment amino acid residues 3-6 of hHBP
GGRK

SEQ ID NO: 219 - protein, 4 aa, Artificial sequence
  Peptide fragment amino acid residues 4-7 of hHBP
GRKA

SEQ ID NO: 220 - protein, 4 aa, Artificial sequence
  Peptide fragment amino acid residues 5-8 of hHBP
RKAR

SEQ ID NO: 221 - protein, 4 aa, Artificial sequence
  Peptide fragment amino acid residues  6-9 of hHBP
KARP

SEQ ID NO: 222 - protein, 4 aa, Artificial sequence
  Peptide fragment amino acid residues 3-6 of pHBP
GGRR

SEQ ID NO: 223 - protein, 4 aa, Artificial sequence
  Peptide fragment amino acid residues 4-7 of pHBP
GRRA

SEQ ID NO: 224 - protein, 4 aa, Artificial sequence
  Peptide fragment amino acid residues 5-8 of pHBP
RRAQ

SEQ ID NO: 225 - protein, 4 aa, Artificial sequence
  Peptide fragment amino acid residues 6-9 of pHBP
RAQP

SEQ ID NO: 226 - protein, 4 aa, Artificial sequence
  Peptide fragment amino acid residues 7-10 of pHBP
AQPQ

SEQ ID NO: 227 - protein, 4 aa, Artificial sequence
  Peptide fragment amino acid residues 8-11 of pHBP
QPQE

SEQ ID NO: 228 - protein, 4 aa, Artificial sequence
  Peptide fragment amino acid residues 9-12 of pHBP
PQEF

SEQ ID NO: 229 - protein, 4 aa, Artificial sequence
  Peptide fragment amino acid residues 10-13 of pHBP
QEFP

SEQ ID NO: 230 - protein, 4 aa, Artificial sequence
  Peptide fragment amino acid residues 11-14 of pHBP
EFPF

SEQ ID NO: 231 - protein, 4 aa, Artificial sequence
  Peptide fragment amino acid residues 12-15 of pHBP
FPFL

SEQ ID NO: 232 - protein, 4 aa, Artificial sequence
  Peptide fragment amino acid residues 13-16 of pHBP
PFLA

SEQ ID NO: 233 - protein, 4 aa, Artificial sequence
  Peptide fragment amino acid residues 21-24 of pHBP
QGRP

SEQ ID NO: 234 - protein, 4 aa, Artificial sequence
  Peptide fragment amino acid residues 22-25 of pHBP
GRPF

SEQ ID NO: 235 - protein, 4 aa, Artificial sequence
  Peptide fragment amino acid residues 23-26 of pHBP
RPFC

SEQ ID NO: 236 - protein, 4 aa, Artificial sequence
  Peptide fragment amino acid residues 24-27 of pHBP
PFCA

SEQ ID NO: 237 - protein, 4 aa, Artificial sequence
  Peptide fragment amino acid residues 25-28 of pHBP
FCAG

SEQ ID NO: 238 - protein, 4 aa, Artificial sequence
  Peptide fragment amino acid residues 26-29 of pHBP
CAGA

SEQ ID NO: 239 - protein, 4 aa, Artificial sequence
  Peptide fragment amino acid residues 27-30 of pHBP
AGAL

SEQ ID NO: 240 - protein, 4 aa, Artificial sequence
  Peptide fragment amino acid residues 28-31 of pHBP
GALV

SEQ ID NO: 241 - protein, 4 aa, Artificial sequence
  Peptide fragment amino acid residues 29-32 of pHBP
ALVH

SEQ ID NO: 242 - protein, 4 aa, Artificial sequence
  Peptide fragment amino acid residues 30-33 of pHBP
LVHP

SEQ ID NO: 243 - protein, 4 aa, Artificial sequence
  Peptide fragment amino acid residues 31-34 of pHBP
VHPR

SEQ ID NO: 244 - protein, 4 aa, Artificial sequence
  Peptide fragment amino acid residues 32-35 of pHBP
HPRF

SEQ ID NO: 245 - protein, 4 aa, Artificial sequence
  Peptide fragment amino acid residues 17-20 of pHBP
SIQK

SEQ ID NO: 246 - protein, 4 aa, Artificial sequence
  Peptide fragment amino acid residues 34-37 of pHBP
RFVL

SEQ ID NO: 247 - protein, 4 aa, Artificial sequence
  Peptide fragment amino acid residues 35-38 of pHBP
FVLT

SEQ ID NO: 248 - protein, 4 aa, Artificial sequence
  Peptide fragment amino acid residues 36-39 of pHBP
VLTA

SEQ ID NO: 249 - protein, 4 aa, Artificial sequence
  Peptide fragment amino acid residues 37-40 of pHBP
LTAA

SEQ ID NO: 250 - protein, 4 aa, Artificial sequence
  Peptide fragment amino acid residues 41-44 of pHBP
SCFR

SEQ ID NO: 251 - protein, 4 aa, Artificial sequence
  Peptide fragment amino acid residues 42-45 of pHBP
CFRG

SEQ ID NO: 252 - protein, 4 aa, Artificial sequence
  Peptide fragment amino acid residues 43-46 of pHBP
FRGK

SEQ ID NO: 253 - protein, 4 aa, Artificial sequence
  Peptide fragment amino acid residues 44-47 of pHBP
RGKN

SEQ ID NO: 254 - protein, 4 aa, Artificial sequence
  Peptide fragment amino acid residues 45-48 of pHBP
GKNS

SEQ ID NO: 255 - protein, 4 aa, Artificial sequence
  Peptide fragment amino acid residues 46-49 of pHBP
KNSG

SEQ ID NO: 256 - protein, 4 aa, Artificial sequence
  Peptide fragment amino acid residues 47-50 of pHBP
NSGS

SEQ ID NO: 257 - protein, 4 aa, Artificial sequence
  Peptide fragment amino acid residues 48-51 of pHBP
SGSA

SEQ ID NO: 258 - protein, 4 aa, Artificial sequence
  Peptide fragment amino acid residues 49-52 of pHBP
GSAS

SEQ ID NO: 259 - protein, 4 aa, Artificial sequence
  Peptide fragment amino acid residues 50-53 of pHBP
SASV

SEQ ID NO: 260 - protein, 4 aa, Artificial sequence
  Peptide fragment amino acid residues 51-54 of pHBP
ASVV

SEQ ID NO: 261 - protein, 4 aa, Artificial sequence
  Peptide fragment amino acid residues 52-55 of pHBP
SVVL

SEQ ID NO: 262 - protein, 4 aa, Artificial sequence
  Peptide fragment amino acid residues 59-62 of pHBP
DLRQ

SEQ ID NO: 263 - protein, 4 aa, Artificial sequence
  Peptide fragment amino acid residues 60-63 of pHBP
LRQQ

SEQ ID NO: 264 - protein, 4 aa, Artificial sequence
  Peptide fragment amino acid residues 61-64 of pHBP
RQQE

SEQ ID NO: 265 - protein, 4 aa, Artificial sequence
  Peptide fragment amino acid residues 62-65 of pHBP
QQEQ

SEQ ID NO: 266 - protein, 4 aa, Artificial sequence
  Peptide fragment amino acid residues 63-66 of pHBP
QEQS

SEQ ID NO: 267 - protein, 4 aa, Artificial sequence
  Peptide fragment amino acid residues 64-67 of pHBP
EQSR

SEQ ID NO: 268 - protein, 4 aa, Artificial sequence
  Peptide fragment amino acid residues 18-21 of pHBP
IQKQ

SEQ ID NO: 269 - protein, 4 aa, Artificial sequence
  Peptide fragment amino acid residues 70-73 of pHBP
FSIR

SEQ ID NO: 270 - protein, 4 aa, Artificial sequence
  Peptide fragment amino acid residues 71-74 of pHBP
SIRS

SEQ ID NO: 271 - protein, 4 aa, Artificial sequence
  Peptide fragment amino acid residues 72-75 of pHBP
IRSI

SEQ ID NO: 272 - protein, 4 aa, Artificial sequence
  Peptide fragment amino acid residues 73-76 of pHBP
RSIS

SEQ ID NO: 273 - protein, 4 aa, Artificial sequence
  Peptide fragment amino acid residues 74-77 of pHBP
SISQ

SEQ ID NO: 274 - protein, 4 aa, Artificial sequence
  Peptide fragment amino acid residues 75-78 of pHBP
ISQN

SEQ ID NO: 275 - protein, 4 aa, Artificial sequence
  Peptide fragment amino acid residues 76-79 of pHBP
SQNG

SEQ ID NO: 276 - protein, 4 aa, Artificial sequence
  Peptide fragment amino acid residues 77-80 of pHBP
QNGY

SEQ ID NO: 277 - protein, 4 aa, Artificial sequence
  Peptide fragment amino acid residues 80-83 of pHBP
YDPR

SEQ ID NO: 278 - protein, 4 aa, Artificial sequence
  Peptide fragment amino acid residues 81-84 of pHBP
DPRQ

SEQ ID NO: 279 - protein, 4 aa, Artificial sequence
  Peptide fragment amino acid residues 82-85 of pHBP
PRQN

SEQ ID NO: 280 - protein, 4 aa, Artificial sequence
  Peptide fragment amino acid residues 83-86 of pHBP
RQNL

SEQ ID NO: 281 - protein, 4 aa, Artificial sequence
  Peptide fragment amino acid residues 86-89 of pHBP
LNDV

SEQ ID NO: 282 - protein, 4 aa, Artificial sequence
  Peptide fragment amino acid residues 87-90 of pHBP
NDVL

SEQ ID NO: 283 - protein, 4 aa, Artificial sequence
  Peptide fragment amino acid residues 88-91 of pHBP
DVLL

SEQ ID NO: 284 - protein, 4 aa, Artificial sequence
  Peptide fragment amino acid residues 89-92 of pHBP
VLLL

SEQ ID NO: 285 - protein, 4 aa, Artificial sequence
  Peptide fragment amino acid residues 90-93 of pHBP
LLLQ

SEQ ID NO: 286 - protein, 4 aa, Artificial sequence
  Peptide fragment amino acid residues 96-99 of pHBP
REAR

SEQ ID NO: 287 - protein, 4 aa, Artificial sequence
  Peptide fragment amino acid residues 97-100 of pHBP
EARL

SEQ ID NO: 288 - protein, 4 aa, Artificial sequence
  Peptide fragment amino acid residues 98-101 of pHBP
ARLT

SEQ ID NO: 289 - protein, 4 aa, Artificial sequence
  Peptide fragment amino acid residues 99-102 of pHBP
RLTP

SEQ ID NO: 290 - protein, 4 aa, Artificial sequence
  Peptide fragment amino acid residues 100-103 of pHBP
LTPS

SEQ ID NO: 291 - protein, 4 aa, Artificial sequence
  Peptide fragment amino acid residues 101-104 of pHBP
TPSV

SEQ ID NO: 292 - protein, 4 aa, Artificial sequence
  Peptide fragment amino acid residues 102-105 of pHBP
PSVA

SEQ ID NO: 293 - protein, 4 aa, Artificial sequence
  Peptide fragment amino acid residues 103-106 of pHBP
SVAL

SEQ ID NO: 294 - protein, 4 aa, Artificial sequence
  Peptide fragment amino acid residues 104-107 of pHBP
VALV

SEQ ID NO: 295 - protein, 4 aa, Artificial sequence
  Peptide fragment amino acid residues 105-108 of pHBP
ALVP

SEQ ID NO: 296 - protein, 4 aa, Artificial sequence
  Peptide fragment amino acid residues 106-109 of pHBP
LVPL

SEQ ID NO: 297 - protein, 4 aa, Artificial sequence
  Peptide fragment amino acid residues 107-110 of pHBP
VPLP

SEQ ID NO: 298 - protein, 4 aa, Artificial sequence
  Peptide fragment amino acid residues 108-111 of pHBP
PLPP

SEQ ID NO: 299 - protein, 4 aa, Artificial sequence
  Peptide fragment amino acid residues 109-112 of pHBP
LPPQ

SEQ ID NO: 300 - protein, 4 aa, Artificial sequence
  Peptide fragment amino acid residues 110-113 of pHBP
PPQN

SEQ ID NO: 301 - protein, 4 aa, Artificial sequence
  Peptide fragment amino acid residues 111-114 of pHBP
PQNA

SEQ ID NO: 302 - protein, 4 aa, Artificial sequence
  Peptide fragment amino acid residues 112-115 of pHBP
AGTN

SEQ ID NO: 303 - protein, 4 aa, Artificial sequence
  Peptide fragment amino acid residues 113-116 of pHBP
GTNC

SEQ ID NO: 304 - protein, 4 aa, Artificial sequence
  Peptide fragment amino acid residues 114-117 of pHBP
TNCQ

SEQ ID NO: 305 - protein, 4 aa, Artificial sequence
  Peptide fragment amino acid residues 121-124 of pHBP
NCQV

SEQ ID NO: 306 - protein, 4 aa, Artificial sequence
  Peptide fragment amino acid residues 126-129 of pHBP
GWGT

SEQ ID NO: 307 - protein, 4 aa, Artificial sequence
  Peptide fragment amino acid residues 127-130 of pHBP
WGTQ

SEQ ID NO: 308 - protein, 4 aa, Artificial sequence
  Peptide fragment amino acid residues 128-131 of pHBP
GTQR

SEQ ID NO: 309 - protein, 4 aa, Artificial sequence
  Peptide fragment amino acid residues 129-132 of pHBP
TQRL

SEQ ID NO: 310 - protein, 4 aa, Artificial sequence
  Peptide fragment amino acid residues 130-133 of pHBP
QRLR

SEQ ID NO: 311 - protein, 4 aa, Artificial sequence
  Peptide fragment amino acid residues 131-134 of pHBP
RLRR

SEQ ID NO: 312 - protein, 4 aa, Artificial sequence
  Peptide fragment amino acid residues 132-135 of pHBP
LRRL

SEQ ID NO: 313 - protein, 4 aa, Artificial sequence
  Peptide fragment amino acid residues 133-136 of pHBP
RRLF

SEQ ID NO: 314 - protein, 4 aa, Artificial sequence
  Peptide fragment amino acid residues 134-137 of pHBP
RLFS

SEQ ID NO: 315 - protein, 4 aa, Artificial sequence
  Peptide fragment amino acid residues 135-138 of pHBP
LFSR

SEQ ID NO: 316 - protein, 4 aa, Artificial sequence
  Peptide fragment amino acid residues 136-139 of pHBP
FSRF

SEQ ID NO: 317 - protein, 4 aa, Artificial sequence
  Peptide fragment amino acid residues 139-142 of pHBP
FPRV

SEQ ID NO: 318 - protein, 4 aa, Artificial sequence
  Peptide fragment amino acid residues 140-143 of pHBP
PRVL

SEQ ID NO: 319 - protein, 4 aa, Artificial sequence
  Peptide fragment amino acid residues 141-144 of pHBP
RVLN

SEQ ID NO: 320 - protein, 4 aa, Artificial sequence
  Peptide fragment amino acid residues 142-145 of pHBP
VLNV

SEQ ID NO: 321 - protein, 4 aa, Artificial sequence
  Peptide fragment amino acid residues 143-146 of pHBP
LNVT

SEQ ID NO: 322 - protein, 4 aa, Artificial sequence
  Peptide fragment amino acid residues 146-149 of pHBP
TVTS

SEQ ID NO: 323 - protein, 4 aa, Artificial sequence
  Peptide fragment amino acid residues 147-150 of pHBP
VTSN

SEQ ID NO: 324 - protein, 4 aa, Artificial sequence
  Peptide fragment amino acid residues 148-151 of pHBP
TSNP

SEQ ID NO: 325 - protein, 4 aa, Artificial sequence
  Peptide fragment amino acid residues 149-152 of pHBP
SNPC

SEQ ID NO: 326 - protein, 4 aa, Artificial sequence
  Peptide fragment amino acid residues 150-153 of pHBP
NPCL

SEQ ID NO: 327 - protein, 4 aa, Artificial sequence
  Peptide fragment amino acid residues 151-154 of pHBP
PCLP

SEQ ID NO: 328 - protein, 4 aa, Artificial sequence
  Peptide fragment amino acid residues 152-155 of pHBP
CLPR

SEQ ID NO: 329 - protein, 4 aa, Artificial sequence
  Peptide fragment amino acid residues 153-156 of pHBP
LPRD

SEQ ID NO: 330 - protein, 4 aa, Artificial sequence
  Peptide fragment amino acid residues 154-157 of pHBP
PRDM

SEQ ID NO: 331 - protein, 4 aa, Artificial sequence
  Peptide fragment amino acid residues 155-158 of pHBP
RDMC

SEQ ID NO: 332 - protein, 4 aa, Artificial sequence
  Peptide fragment amino acid residues 156-159 of pHBP
DMCI

SEQ ID NO: 333 - protein, 4 aa, Artificial sequence
  Peptide fragment amino acid residues 160 of pHBP
MCIG

SEQ ID NO: 334 - protein, 4 aa, Artificial sequence
  Peptide fragment amino acid residues 158-161 of pHBP
CIGV

SEQ ID NO: 335 - protein, 4 aa, Artificial sequence
  Peptide fragment amino acid residues 159-162 of pHBP
IGVF

SEQ ID NO: 336 - protein, 4 aa, Artificial sequence
  Peptide fragment amino acid residues 160-163 of pHBP
GVFS

SEQ ID NO: 337 - protein, 4 aa, Artificial sequence
  Peptide fragment amino acid residues 161-164 of pHBP
VFSR

SEQ ID NO: 338 - protein, 4 aa, Artificial sequence
  Peptide fragment amino acid residues 162-165 of pHBP
FSRR

SEQ ID NO: 339 - protein, 4 aa, Artificial sequence
  Peptide fragment amino acid residues 163-166 of pHBP
SRRG

SEQ ID NO: 340 - protein, 4 aa, Artificial sequence
  Peptide fragment amino acid residues 164-167 of pHBP
RRGR

SEQ ID NO: 341 - protein, 4 aa, Artificial sequence
  Peptide fragment amino acid residues 165-168 of pHBP
RGRI

SEQ ID NO: 342 - protein, 4 aa, Artificial sequence
  Peptide fragment amino acid residues 166-169 of pHBP
GRIS

SEQ ID NO: 343 - protein, 4 aa, Artificial sequence
  Peptide fragment amino acid residues 167-170 of pHBP
RISQ

SEQ ID NO: 344 - protein, 4 aa, Artificial sequence
  Peptide fragment amino acid residues 168-171 of pHBP
ISQG

SEQ ID NO: 345 - protein, 4 aa, Artificial sequence
  Peptide fragment amino acid residues 169-172 of pHBP
SQGD

SEQ ID NO: 346 - protein, 4 aa, Artificial sequence
  Peptide fragment amino acid residues 170-173 of pHBP
QGDR

SEQ ID NO: 347 - protein, 4 aa, Artificial sequence
  Peptide fragment amino acid residues 171-174 of pHBP
GDRG

SEQ ID NO: 348 - protein, 4 aa, Artificial sequence
  Peptide fragment amino acid residues 172-175 of pHBP
DRGT

SEQ ID NO: 349 - protein, 4 aa, Artificial sequence
  Peptide fragment amino acid residues 173-176 of pHBP
RGTP

SEQ ID NO: 350 - protein, 4 aa, Artificial sequence
  Peptide fragment amino acid residues 177-180 of pHBP
LVCN

SEQ ID NO: 351 - protein, 4 aa, Artificial sequence
  Peptide fragment amino acid residues 178-181 of pHBP
VCNG

SEQ ID NO: 352 - protein, 4 aa, Artificial sequence
  Peptide fragment amino acid residues 179-182 of pHBP
CNGL

SEQ ID NO: 353 - protein, 4 aa, Artificial sequence
  Peptide fragment amino acid residues 180-183 of pHBP
NGLA

SEQ ID NO: 354 - protein, 4 aa, Artificial sequence
  Peptide fragment amino acid residues181-184 of pHBP
GLAQ

SEQ ID NO: 355 - protein, 4 aa, Artificial sequence
  Peptide fragment amino acid residues 182-185 of pHBP
LAQG

SEQ ID NO: 356 - protein, 4 aa, Artificial sequence
  Peptide fragment amino acid residues 183-186 of pHBP
AQGV

SEQ ID NO: 357 - protein, 4 aa, Artificial sequence
  Peptide fragment amino acid residues 184-187 of pHBP
QGVA

SEQ ID NO: 358 - protein, 4 aa, Artificial sequence
  Peptide fragment amino acid residues 187-190 of pHBP
ASFL

SEQ ID NO: 359 - protein, 4 aa, Artificial sequence
  Peptide fragment amino acid residues 188-191 of pHBP
SFLR

SEQ ID NO: 360 - protein, 4 aa, Artificial sequence
  Peptide fragment amino acid residues 189-192 of pHBP
FLRR

SEQ ID NO: 361 - protein, 4 aa, Artificial sequence
  Peptide fragment amino acid residues 19-22 of pHBP
QKQG

SEQ ID NO: 362 - protein, 4 aa, Artificial sequence
  Peptide fragment amino acid residues 191-194 of pHBP
RRRF

SEQ ID NO: 363 - protein, 4 aa, Artificial sequence
  Peptide fragment amino acid residues 192-195 of pHBP
RRFR

SEQ ID NO: 364 - protein, 4 aa, Artificial sequence
  Peptide fragment amino acid residues 193-196 of pHBP
RFRR

SEQ ID NO: 365 - protein, 4 aa, Artificial sequence
  Peptide fragment amino acid residues 194-197 of pHBP
FRRS

SEQ ID NO: 366 - protein, 4 aa, Artificial sequence
  Peptide fragment amino acid residues 195-198 of pHBP
RRSS

SEQ ID NO: 367 - protein, 4 aa, Artificial sequence
  Peptide fragment amino acid residues 196-199 of pHBP
RSSG

SEQ ID NO: 368 - protein, 4 aa, Artificial sequence
  Peptide fragment amino acid residues 197-200 of pHBP
SSGF

SEQ ID NO: 369 - protein, 4 aa, Artificial sequence
  Peptide fragment amino acid residues 198-201 of pHBP
SGFF

SEQ ID NO: 370 - protein, 4 aa, Artificial sequence
  Peptide fragment amino acid residues 199-202 of pHBP
GFFT

SEQ ID NO: 371 - protein, 4 aa, Artificial sequence
  Peptide fragment amino acid residues 20-23 of pHBP
KQGR

SEQ ID NO: 372 - protein, 4 aa, Artificial sequence
  Peptide fragment amino acid residues 206-209 of pHBP
LFRN

SEQ ID NO: 373 - protein, 4 aa, Artificial sequence
  Peptide fragment amino acid residues 207-210 of pHBP
FRNW

SEQ ID NO: 374 - protein, 4 aa, Artificial sequence
  Peptide fragment amino acid residues 208-211 of pHBP
RNWI

SEQ ID NO: 375 - protein, 4 aa, Artificial sequence
  Peptide fragment amino acid residues 209-212 of pHBP
NWID

SEQ ID NO: 376 - protein, 4 aa, Artificial sequence
  Peptide fragment amino acid residues 210-213 of pHBP
WIDS

SEQ ID NO: 377 - protein, 4 aa, Artificial sequence
  Peptide fragment amino acid residues 211-214 of pHBP
IDSV

SEQ ID NO: 378 - protein, 4 aa, Artificial sequence
  Peptide fragment amino acid residues 212-214 of pHBP
DSVL

SEQ ID NO: 379 - protein, 4 aa, Artificial sequence
  Peptide fragment amino acid residues 213-216 of pHBP
SVLN

SEQ ID NO: 380 - protein, 4 aa, Artificial sequence
  Peptide fragment amino acid residues 216-219 of pHBP
NNPP

SEQ ID NO: 381 - protein, 4 aa, Artificial sequence
  Peptide fragment amino acid residues 3-6 of hNLE
GGRR

SEQ ID NO: 382 - protein, 4 aa, Artificial sequence
  Peptide fragment amino acid residues 4-7 of hNLE
GRRA

SEQ ID NO: 383 - protein, 4 aa, Artificial sequence
  Peptide fragment amino acid residues 5-8 of hNLE
RRAR

SEQ ID NO: 384 - protein, 4 aa, Artificial sequence
  Peptide fragment amino acid residues 6-9 of hNLE
RARP

SEQ ID NO: 385 - protein, 4 aa, Artificial sequence
  Peptide fragment amino acid residues 7-10 of hNLE
ARPH

SEQ ID NO: 386 - protein, 4 aa, Artificial sequence
  Peptide fragment amino acid residues 8-11 of hNLE
RPHA

SEQ ID NO: 387 - protein, 4 aa, Artificial sequence
  Peptide fragment amino acid residues 9-12 of hNLE
PHAW

SEQ ID NO: 388 - protein, 4 aa, Artificial sequence
  Peptide fragment amino acid residues 10-13 of hNLE
HAWP

SEQ ID NO: 389 - protein, 4 aa, Artificial sequence
  Peptide fragment amino acid residues 11-14 of hNLE
AWPF

SEQ ID NO: 390 - protein, 4 aa, Artificial sequence
  Peptide fragment amino acid residues 12-15 of hNLE
WPFM

SEQ ID NO: 391 - protein, 4 aa, Artificial sequence
  Peptide fragment amino acid residues 13-16 of hNLE
PFMV

SEQ ID NO: 392 - protein, 4 aa, Artificial sequence
  Peptide fragment amino acid residues 14-17 of hNLE
FMVS

SEQ ID NO: 393 - protein, 4 aa, Artificial sequence
  Peptide fragment amino acid residues 15-18 of hNLE
MVSL

SEQ ID NO: 394 - protein, 4 aa, Artificial sequence
  Peptide fragment amino acid residues 16-19 of hNLE
VSLQ

SEQ ID NO: 395 - protein, 4 aa, Artificial sequence
  Peptide fragment amino acid residues 17-20 of hNLE
SLQL

SEQ ID NO: 396 - protein, 4 aa, Artificial sequence
  Peptide fragment amino acid residues 18-21 of hNLE
LQLR

SEQ ID NO: 397 - protein, 4 aa, Artificial sequence
  Peptide fragment amino acid residues 19-22 of hNLE
QLRG

SEQ ID NO: 398 - protein, 4 aa, Artificial sequence
  Peptide fragment amino acid residues 20-23 of hNLE
LRGG

SEQ ID NO: 399 - protein, 4 aa, Artificial sequence
  Peptide fragment amino acid residues 21-24 of hNLE
RGGH

SEQ ID NO: 400 - protein, 4 aa, Artificial sequence
  Peptide fragment amino acid residues 22-25 of hNLE
GGHF

SEQ ID NO: 401 - protein, 4 aa, Artificial sequence
  Peptide fragment amino acid residues 23-26 of hNLE
GHFC

SEQ ID NO: 402 - protein, 4 aa, Artificial sequence
  Peptide fragment amino acid residues 25-28 of hNLE
FCGA

SEQ ID NO: 403 - protein, 4 aa, Artificial sequence
  Peptide fragment amino acid residues 26-29 of hNLE
CGAT

SEQ ID NO: 404 - protein, 4 aa, Artificial sequence
  Peptide fragment amino acid residues 27-30 of hNLE
GATL

SEQ ID NO: 405 - protein, 4 aa, Artificial sequence
  Peptide fragment amino acid residues 28-31 of hNLE
ATLI

SEQ ID NO: 406 - protein, 4 aa, Artificial sequence
  Peptide fragment amino acid residues 29-32 of hNLE
TLIA

SEQ ID NO: 407 - protein, 4 aa, Artificial sequence
  Peptide fragment amino acid residues 30-33 of hNLE
LIAP

SEQ ID NO: 408 - protein, 4 aa, Artificial sequence
  Peptide fragment amino acid residues 31-34 of hNLE
IAPN

SEQ ID NO: 409 - protein, 4 aa, Artificial sequence
  Peptide fragment amino acid residues 32-35 of hNLE
APNF

SEQ ID NO: 410 - protein, 4 aa, Artificial sequence
  Peptide fragment amino acid residues 32-36 of hNLE
PNFV

SEQ ID NO: 411 - protein, 4 aa, Artificial sequence
  Peptide fragment amino acid residues 34-37 of hNLE
NFVM

SEQ ID NO: 412 - protein, 4 aa, Artificial sequence
  Peptide fragment amino acid residues 35-38 of hNLE
FVMS

SEQ ID NO: 413 - protein, 4 aa, Artificial sequence
  Peptide fragment amino acid residues 36-39 of hNLE
VMSA

SEQ ID NO: 414 - protein, 4 aa, Artificial sequence
  Peptide fragment amino acid residues 37-40 of hNLE
MSAA

SEQ ID NO: 415 - protein, 4 aa, Artificial sequence
  Peptide fragment amino acid residues 38-41 of hNLE
SAAH

SEQ ID NO: 416 - protein, 4 aa, Artificial sequence
  Peptide fragment amino acid residues 39-42 of hNLE
AAHC

SEQ ID NO: 417 - protein, 4 aa, Artificial sequence
  Peptide fragment amino acid residues 40-43 of hNLE
AHCV

SEQ ID NO: 418 - protein, 4 aa, Artificial sequence
  Peptide fragment amino acid residues 41-44 of hNLE
HCVA

SEQ ID NO: 419 - protein, 4 aa, Artificial sequence
  Peptide fragment amino acid residues 42-45 of hNLE
CVAN

SEQ ID NO: 420 - protein, 4 aa, Artificial sequence
  Peptide fragment amino acid residues 43-36 of hNLE
VANV

SEQ ID NO: 421 - protein, 4 aa, Artificial sequence
  Peptide fragment amino acid residues 44-47 of hNLE
ANVN

SEQ ID NO: 422 - protein, 4 aa, Artificial sequence
  Peptide fragment amino acid residues 45-48 of hNLE
NVNV

SEQ ID NO: 423 - protein, 4 aa, Artificial sequence
  Peptide fragment amino acid residues 46-49 of hNLE
VNVR

SEQ ID NO: 424 - protein, 4 aa, Artificial sequence
  Peptide fragment amino acid residues 48-51 of hNLE
VRAV

SEQ ID NO: 425 - protein, 4 aa, Artificial sequence
  Peptide fragment amino acid residues 49-52 of hNLE
RAVR

SEQ ID NO: 426 - protein, 4 aa, Artificial sequence
  Peptide fragment amino acid residues 50-53 of hNLE
AVRV

SEQ ID NO: 427 - protein, 4 aa, Artificial sequence
  Peptide fragment amino acid residues 51-54 of hNLE
VRVV

SEQ ID NO: 428 - protein, 4 aa, Artificial sequence
  Peptide fragment amino acid residues 52-55 of hNLE
RVVL

SEQ ID NO: 429 - protein, 4 aa, Artificial sequence
  Peptide fragment amino acid residues 55-58 of hNLE
LGAH

SEQ ID NO: 430 - protein, 4 aa, Artificial sequence
  Peptide fragment amino acid residues 56-59 of hNLE
GAHN

SEQ ID NO: 431 - protein, 4 aa, Artificial sequence
  Peptide fragment amino acid residues 57-60 of hNLE
AHNL

SEQ ID NO: 432 - protein, 4 aa, Artificial sequence
  Peptide fragment amino acid residues 58-61 of hNLE
HNLS

SEQ ID NO: 433 - protein, 4 aa, Artificial sequence
  Peptide fragment amino acid residues 59-62 of hNLE
NLSR

SEQ ID NO: 434 - protein, 4 aa, Artificial sequence
  Peptide fragment amino acid residues 60-63 of hNLE
LSRR

SEQ ID NO: 435 - protein, 4 aa, Artificial sequence
  Peptide fragment amino acid residues 61-64 of hNLE
SRRE

SEQ ID NO: 436 - protein, 4 aa, Artificial sequence
  Peptide fragment amino acid residues 62-65 of hNLE
RREP

SEQ ID NO: 437 - protein, 4 aa, Artificial sequence
  Peptide fragment amino acid residues 63-66 of hNLE
REPT

SEQ ID NO: 438 - protein, 4 aa, Artificial sequence
  Peptide fragment amino acid residues 64-67 of hNLE
EPTR

SEQ ID NO: 439 - protein, 4 aa, Artificial sequence
  Peptide fragment amino acid residues 65-68 of hNLE
PTRQ

SEQ ID NO: 440 - protein, 4 aa, Artificial sequence
  Peptide fragment amino acid residues 66-69 of hNLE
TRQV

SEQ ID NO: 441 - protein, 4 aa, Artificial sequence
  Peptide fragment amino acid residues 67-70 of hNLE
RQVF

SEQ ID NO: 442 - protein, 4 aa, Artificial sequence
  Peptide fragment amino acid residues 68-71 of hNLE
QVFA

SEQ ID NO: 443 - protein, 4 aa, Artificial sequence
  Peptide fragment amino acid residues 69-72 of hNLE
VFAV

SEQ ID NO: 444 - protein, 4 aa, Artificial sequence
  Peptide fragment amino acid residues 70-73 of hNLE
FAVQ

SEQ ID NO: 445 - protein, 4 aa, Artificial sequence
  Peptide fragment amino acid residues 71-74 of hNLE
AVQR

SEQ ID NO: 446 - protein, 4 aa, Artificial sequence
  Peptide fragment amino acid residues 72-75 of hNLE
VQRI

SEQ ID NO: 447 - protein, 4 aa, Artificial sequence
  Peptide fragment amino acid residues 73-76 of hNLE
QRIF

SEQ ID NO: 448 - protein, 4 aa, Artificial sequence
  Peptide fragment amino acid residues 74-77 of hNLE
RIFE

SEQ ID NO: 449 - protein, 4 aa, Artificial sequence
  Peptide fragment amino acid residues 75-78 of hNLE
IFED

SEQ ID NO: 450 - protein, 4 aa, Artificial sequence
  Peptide fragment amino acid residues 76-79 of hNLE
FEDG

SEQ ID NO: 451 - protein, 4 aa, Artificial sequence
  Peptide fragment amino acid residues 77-80 of hNLE
EDGY

SEQ ID NO: 452 - protein, 4 aa, Artificial sequence
  Peptide fragment amino acid residues 78-81 of hNLE
DGYD

SEQ ID NO: 453 - protein, 4 aa, Artificial sequence
  Peptide fragment amino acid residues 80-83 of hNLE
YDPV

SEQ ID NO: 454 - protein, 4 aa, Artificial sequence
  Peptide fragment amino acid residues 81-84 of hNLE
DPVN

SEQ ID NO: 455 - protein, 4 aa, Artificial sequence
  Peptide fragment amino acid residues 82-84 of hNLE
PVNL

SEQ ID NO: 456 - protein, 4 aa, Artificial sequence
  Peptide fragment amino acid residues 83-86 of hNLE
VNLL

SEQ ID NO: 457 - protein, 4 aa, Artificial sequence
  Peptide fragment amino acid residues 84-87 of hNLE
NLLN

SEQ ID NO: 458 - protein, 4 aa, Artificial sequence
  Peptide fragment amino acid residues 85-88 of hNLE
LLND

SEQ ID NO: 459 - protein, 4 aa, Artificial sequence
  Peptide fragment amino acid residues 86-89 of hNLE
LNDI

SEQ ID NO: 460 - protein, 4 aa, Artificial sequence
  Peptide fragment amino acid residues 87-90 of hNLE
NDIV

SEQ ID NO: 461 - protein, 4 aa, Artificial sequence
  Peptide fragment amino acid residues 88-91 of hNLE
DIVI

SEQ ID NO: 462 - protein, 4 aa, Artificial sequence
  Peptide fragment amino acid residues 89-92 of hNLE
IVIL

SEQ ID NO: 463 - protein, 4 aa, Artificial sequence
  Peptide fragment amino acid residues 90-93 of hNLE
VILQ

SEQ ID NO: 464 - protein, 4 aa, Artificial sequence
  Peptide fragment amino acid residues 91-94 of hNLE
ILQL

SEQ ID NO: 465 - protein, 4 aa, Artificial sequence
  Peptide fragment amino acid residues 92-95 of hNLE
LQLN

SEQ ID NO: 466 - protein, 4 aa, Artificial sequence
  Peptide fragment amino acid residues 93-96 of hNLE
QLNG

SEQ ID NO: 467 - protein, 4 aa, Artificial sequence
  Peptide fragment amino acid residues 94-97 of hNLE
LNGS

SEQ ID NO: 468 - protein, 4 aa, Artificial sequence
  Peptide fragment amino acid residues 95-98 of hNLE
NGSA

SEQ ID NO: 469 - protein, 4 aa, Artificial sequence
  Peptide fragment amino acid residues 96-99 of hNLE
GSAT

SEQ ID NO: 470 - protein, 4 aa, Artificial sequence
  Peptide fragment amino acid residues 97-100 of hNLE
SATI

SEQ ID NO: 471 - protein, 4 aa, Artificial sequence
  Peptide fragment amino acid residues 98-101 of hNLE
ATIN

SEQ ID NO: 472 - protein, 4 aa, Artificial sequence
  Peptide fragment amino acid residues 99-102 of hNLE
TINP

SEQ ID NO: 473 - protein, 4 aa, Artificial sequence
  Peptide fragment amino acid residues 100-103 of hNLE
INPS

SEQ ID NO: 474 - protein, 4 aa, Artificial sequence
  Peptide fragment amino acid residues 101-104 of hNLE
NPSV

SEQ ID NO: 475 - protein, 4 aa, Artificial sequence
  Peptide fragment amino acid residues 102-105 of hNLE
PSVA

SEQ ID NO: 476 - protein, 4 aa, Artificial sequence
  Peptide fragment amino acid residues 103-106 of hNLE
SVAL

SEQ ID NO: 477 - protein, 4 aa, Artificial sequence
  Peptide fragment amino acid residues 104-107 of hNLE
VALV

SEQ ID NO: 478 - protein, 4 aa, Artificial sequence
  Peptide fragment amino acid residues 105-108 of hNLE
ALVP

SEQ ID NO: 479 - protein, 4 aa, Artificial sequence
  Peptide fragment amino acid residues 106-109 of hNLE
LVPL

SEQ ID NO: 480 - protein, 4 aa, Artificial sequence
  Peptide fragment amino acid residues 107-110 of hNLE
VPLP

SEQ ID NO: 481 - protein, 4 aa, Artificial sequence
  Peptide fragment amino acid residues 108-111 of hNLE
PLPA

SEQ ID NO: 482 - protein, 4 aa, Artificial sequence
  Peptide fragment amino acid residues 109-112 of hNLE
LPAQ

SEQ ID NO: 483 - protein, 4 aa, Artificial sequence
  Peptide fragment amino acid residues 110-113 of hNLE
PAQG

SEQ ID NO: 484 - protein, 4 aa, Artificial sequence
  Peptide fragment amino acid residues 111-114 of hNLE
AQGR

SEQ ID NO: 485 - protein, 4 aa, Artificial sequence
  Peptide fragment amino acid residues 112-115 of hNLE
QGRR

SEQ ID NO: 486 - protein, 4 aa, Artificial sequence
  Peptide fragment amino acid residues 113-116 of hNLE
GRRL

SEQ ID NO: 487 - protein, 4 aa, Artificial sequence
  Peptide fragment amino acid residues 114-117 of hNLE
RRLG

SEQ ID NO: 488 - protein, 4 aa, Artificial sequence
  Peptide fragment amino acid residues 115-118 of hNLE
RLGN

SEQ ID NO: 489 - protein, 4 aa, Artificial sequence
  Peptide fragment amino acid residues 116-119 of hNLE
LGNG

SEQ ID NO: 490 - protein, 4 aa, Artificial sequence
  Peptide fragment amino acid residues 117-120 of hNLE
GNGV

SEQ ID NO: 491 - protein, 4 aa, Artificial sequence
  Peptide fragment amino acid residues 118-121 of hNLE
NGVQ

SEQ ID NO: 492 - protein, 4 aa, Artificial sequence
  Peptide fragment amino acid residues 119-121 of hNLE
GVQC

SEQ ID NO: 493 - protein, 4 aa, Artificial sequence
  Peptide fragment amino acid residues 120-123 of hNLE
VQCL

SEQ ID NO: 494 - protein, 4 aa, Artificial sequence
  Peptide fragment amino acid residues 121-124 of hNLE
QCLA

SEQ ID NO: 495 - protein, 4 aa, Artificial sequence
  Peptide fragment amino acid residues 122-125 of hNLE
CLAM

SEQ ID NO: 496 - protein, 4 aa, Artificial sequence
  Peptide fragment amino acid residues 123-126 of hNLE
LAMG

SEQ ID NO: 497 - protein, 4 aa, Artificial sequence
  Peptide fragment amino acid residues 124-127 of hNLE
AMGW

SEQ ID NO: 498 - protein, 4 aa, Artificial sequence
  Peptide fragment amino acid residues 125-128 of hNLE
MGWG

SEQ ID NO: 499 - protein, 4 aa, Artificial sequence
  Peptide fragment amino acid residues 126-129 of hNLE
GWGL

SEQ ID NO: 500 - protein, 4 aa, Artificial sequence
  Peptide fragment amino acid residues 127-130 of hNLE
WGLL

SEQ ID NO: 501 - protein, 4 aa, Artificial sequence
  Peptide fragment amino acid residues 47-50 of hNLE
NVRA

SEQ ID NO: 502 - protein, 4 aa, Artificial sequence
  Peptide fragment amino acid residues 128-131 of hNLE
GLLG

SEQ ID NO: 503 - protein, 4 aa, Artificial sequence
  Peptide fragment amino acid residues 129-132 of hNLE
LLGR

SEQ ID NO: 504 - protein, 4 aa, Artificial sequence
  Peptide fragment amino acid residues 130-133 of hNLE
LGRN

SEQ ID NO: 505 - protein, 4 aa, Artificial sequence
  Peptide fragment amino acid residues 131-134 of hNLE
GRNR

SEQ ID NO: 506 - protein, 4 aa, Artificial sequence
  Peptide fragment amino acid residues 132-135 of hNLE
RNRG

SEQ ID NO: 507 - protein, 4 aa, Artificial sequence
  Peptide fragment amino acid residues 133-136 of hNLE
NRGI

SEQ ID NO: 508 - protein, 4 aa, Artificial sequence
  Peptide fragment amino acid residues 134-137 of hNLE
RGIA

SEQ ID NO: 509 - protein, 4 aa, Artificial sequence
  Peptide fragment amino acid residues 135-138 of hNLE
GIAS

SEQ ID NO: 510 - protein, 4 aa, Artificial sequence
  Peptide fragment amino acid residues 136-139 of hNLE
IASV

SEQ ID NO: 511 - protein, 4 aa, Artificial sequence
  Peptide fragment amino acid residues 137-140 of hNLE
ASVL

SEQ ID NO: 512 - protein, 4 aa, Artificial sequence
  Peptide fragment amino acid residues 138-141 of hNLE
SVLQ

SEQ ID NO: 513 - protein, 4 aa, Artificial sequence
  Peptide fragment amino acid residues 139-142 of hNLE
VLQE

SEQ ID NO: 514 - protein, 4 aa, Artificial sequence
  Peptide fragment amino acid residues 140-143 of hNLE
LQEL

SEQ ID NO: 515 - protein, 4 aa, Artificial sequence
  Peptide fragment amino acid residues 141-144 of hNLE
QELN

SEQ ID NO: 516 - protein, 4 aa, Artificial sequence
  Peptide fragment amino acid residues 142-145 of hNLE
ELNV

SEQ ID NO: 517 - protein, 4 aa, Artificial sequence
  Peptide fragment amino acid residues 143-146 of hNLE
LNVT

SEQ ID NO: 518 - protein, 4 aa, Artificial sequence
  Peptide fragment amino acid residues 145-148 of hNLE
VTVV

SEQ ID NO: 519 - protein, 4 aa, Artificial sequence
  Peptide fragment amino acid residues 146-149 of hNLE
TVVT

SEQ ID NO: 520 - protein, 4 aa, Artificial sequence
  Peptide fragment amino acid residues 147-150 of hNLE
VVTS

SEQ ID NO: 521 - protein, 4 aa, Artificial sequence
  Peptide fragment amino acid residues 148-151 of hNLE
VTSL

SEQ ID NO: 522 - protein, 4 aa, Artificial sequence
  Peptide fragment amino acid residues 149-152 of hNLE
TSLC

SEQ ID NO: 523 - protein, 4 aa, Artificial sequence
  Peptide fragment amino acid residues 150-153 of hNLE
SLCR

SEQ ID NO: 524 - protein, 4 aa, Artificial sequence
  Peptide fragment amino acid residues 151-154 of hNLE
LCRR

SEQ ID NO: 525 - protein, 4 aa, Artificial sequence
  Peptide fragment amino acid residues 152-155 of hNLE
CRRS

SEQ ID NO: 526 - protein, 4 aa, Artificial sequence
  Peptide fragment amino acid residues 153-156 of hNLE
RRSN

SEQ ID NO: 527 - protein, 4 aa, Artificial sequence
  Peptide fragment amino acid residues 154-157 of hNLE
RSNV

SEQ ID NO: 528 - protein, 4 aa, Artificial sequence
  Peptide fragment amino acid residues 155-158 of hNLE
SNVC

SEQ ID NO: 529 - protein, 4 aa, Artificial sequence
  Peptide fragment amino acid residues 157-160 of hNLE
VCTL

SEQ ID NO: 530 - protein, 4 aa, Artificial sequence
  Peptide fragment amino acid residues 158-161 of hNLE
CTLV

SEQ ID NO: 531 - protein, 4 aa, Artificial sequence
  Peptide fragment amino acid residues 159-162 of hNLE
TLVR

SEQ ID NO: 532 - protein, 4 aa, Artificial sequence
  Peptide fragment amino acid residues 160-163 of hNLE
LVRG

SEQ ID NO: 533 - protein, 4 aa, Artificial sequence
  Peptide fragment amino acid residues 161-164 of hNLE
VRGR

SEQ ID NO: 534 - protein, 4 aa, Artificial sequence
  Peptide fragment amino acid residues 162-165 of hNLE
RGRR

SEQ ID NO: 535 - protein, 4 aa, Artificial sequence
  Peptide fragment amino acid residues 163-166 of hNLE
GRRG

SEQ ID NO: 536 - protein, 4 aa, Artificial sequence
  Peptide fragment amino acid residues 164-167 of hNLE
RRGR

SEQ ID NO: 537 - protein, 4 aa, Artificial sequence
  Peptide fragment amino acid residues 165-166 of hNLE
RGRI

SEQ ID NO: 538 - protein, 4 aa, Artificial sequence
  Peptide fragment amino acid residues 166-169 of hNLE
GRIS

SEQ ID NO: 539 - protein, 4 aa, Artificial sequence
  Peptide fragment amino acid residues 167-170 of hNLE
RISQ

SEQ ID NO: 540 - protein, 4 aa, Artificial sequence
  Peptide fragment amino acid residues 168-171 of hNLE
ISQG

SEQ ID NO: 541 - protein, 4 aa, Artificial sequence
  Peptide fragment amino acid residues 169-172 of hNLE
SQGD

SEQ ID NO: 542 - protein, 4 aa, Artificial sequence
  Peptide fragment amino acid residues 170-173 of hNLE
QGDS

SEQ ID NO: 543 - protein, 4 aa, Artificial sequence
  Peptide fragment amino acid residues 171-174 of hNLE
GDSG

SEQ ID NO: 544 - protein, 4 aa, Artificial sequence
  Peptide fragment amino acid residues 172-174 of hNLE
DSGT

SEQ ID NO: 545 - protein, 4 aa, Artificial sequence
  Peptide fragment amino acid residues 173-176 of hNLE
SGTP

SEQ ID NO: 546 - protein, 4 aa, Artificial sequence
  Peptide fragment amino acid residues 177-180 of hNLE
LVCN

SEQ ID NO: 547 - protein, 4 aa, Artificial sequence
  Peptide fragment amino acid residues 178-181 of hNLE
VCNG

SEQ ID NO: 548 - protein, 4 aa, Artificial sequence
  Peptide fragment amino acid residues 179-182 of hNLE
CNGL

SEQ ID NO: 549 - protein, 4 aa, Artificial sequence
  Peptide fragment amino acid residues 180-183 of hNLE
NGLI

SEQ ID NO: 550 - protein, 4 aa, Artificial sequence
  Peptide fragment amino acid residues 181-184 of hNLE
GLIH

SEQ ID NO: 551 - protein, 4 aa, Artificial sequence
  Peptide fragment amino acid residues 182-185 of hNLE
LIHG

SEQ ID NO: 552 - protein, 4 aa, Artificial sequence
  Peptide fragment amino acid residues 183-186 of hNLE
IHGI

SEQ ID NO: 553 - protein, 4 aa, Artificial sequence
  Peptide fragment amino acid residues 184-187 of hNLE
HGIA

SEQ ID NO: 554 - protein, 4 aa, Artificial sequence
  Peptide fragment amino acid residues 185-188 of hNLE
GIAS

SEQ ID NO: 555 - protein, 4 aa, Artificial sequence
  Peptide fragment amino acid residues 186-189 of hNLE
IASF

SEQ ID NO: 556 - protein, 4 aa, Artificial sequence
  Peptide fragment amino acid residues 187-190 of hNLE
ASFV

SEQ ID NO: 557 - protein, 4 aa, Artificial sequence
  Peptide fragment amino acid residues 188-191 of hNLE
SFVR

SEQ ID NO: 558 - protein, 4 aa, Artificial sequence
  Peptide fragment amino acid residues 189-192 of hNLE
FVRG

SEQ ID NO: 559 - protein, 4 aa, Artificial sequence
  Peptide fragment amino acid residues 190-103 of hNLE
VRGG

SEQ ID NO: 560 - protein, 4 aa, Artificial sequence
  Peptide fragment amino acid residues 191-194 of hNLE
RGGC

SEQ ID NO: 561 - protein, 4 aa, Artificial sequence
  Peptide fragment amino acid residues 192-195 of hNLE
GGCA

SEQ ID NO: 562 - protein, 4 aa, Artificial sequence
  Peptide fragment amino acid residues 193-196 of hNLE
GCAS

SEQ ID NO: 563 - protein, 4 aa, Artificial sequence
  Peptide fragment amino acid residues 194-197 of hNLE
CASG

SEQ ID NO: 564 - protein, 4 aa, Artificial sequence
  Peptide fragment amino acid residues 195-198 of hNLE
ASGL

SEQ ID NO: 565 - protein, 4 aa, Artificial sequence
  Peptide fragment amino acid residues 196-199 of hNLE
SGLY

SEQ ID NO: 566 - protein, 4 aa, Artificial sequence
  Peptide fragment amino acid residues 197-200 of hNLE
GLYP

SEQ ID NO: 567 - protein, 4 aa, Artificial sequence
  Peptide fragment amino acid residues 198-201 of hNLE
LYPD

SEQ ID NO: 568 - protein, 4 aa, Artificial sequence
  Peptide fragment amino acid residues 199-202 of hNLE
YPDA

SEQ ID NO: 569 - protein, 4 aa, Artificial sequence
  Peptide fragment amino acid residues 200-203 of hNLE
PDAF

SEQ ID NO: 570 - protein, 4 aa, Artificial sequence
  Peptide fragment amino acid residues 201-204 of hNLE
DAFA

SEQ ID NO: 571 - protein, 4 aa, Artificial sequence
  Peptide fragment amino acid residues 202-205 of hNLE
AFAP

SEQ ID NO: 572 - protein, 4 aa, Artificial sequence
  Peptide fragment amino acid residues 203-206 of hNLE
FAPV

SEQ ID NO: 573 - protein, 4 aa, Artificial sequence
  Peptide fragment amino acid residues 204-207 of hNLE
APVA

SEQ ID NO: 574 - protein, 4 aa, Artificial sequence
  Peptide fragment amino acid residues 205-208 of hNLE
PVAQ

SEQ ID NO: 575 - protein, 4 aa, Artificial sequence
  Peptide fragment amino acid residues 206-209 of hNLE
VAQF

SEQ ID NO: 576 - protein, 4 aa, Artificial sequence
  Peptide fragment amino acid residues 207-210 of hNLE
AQFV

SEQ ID NO: 577 - protein, 4 aa, Artificial sequence
  Peptide fragment amino acid residues 208-211 of hNLE
QFVN

SEQ ID NO: 578 - protein, 4 aa, Artificial sequence
  Peptide fragment amino acid residues 209-212 of hNLE
FVNW

SEQ ID NO: 579 - protein, 4 aa, Artificial sequence
  Peptide fragment amino acid residues 210-212 of hNLE
VNWI

SEQ ID NO: 580 - protein, 4 aa, Artificial sequence
  Peptide fragment amino acid residues 211-214 of hNLE
NWID

SEQ ID NO: 581 - protein, 4 aa, Artificial sequence
  Peptide fragment amino acid residues 212-215 of hNLE
WIDS

SEQ ID NO: 582 - protein, 4 aa, Artificial sequence
  Peptide fragment amino acid residues 213-216 of hNLE
IDSI

SEQ ID NO: 583 - protein, 4 aa, Artificial sequence
  Peptide fragment amino acid residues 214-217 of hNLE
DSII

SEQ ID NO: 584 - protein, 4 aa, Artificial sequence
  Peptide fragment amino acid residues 214-218 of hNLE
SIIQ

SEQ ID NO: 585 - protein, 4 aa, Artificial sequence
  Peptide fragment amino acid residues 43-46 of pHBP
PRFV

SEQ ID NO: 586 - protein, 4 aa, Artificial sequence
  Peptide fragment amino acid residues 190-194 of pHBP
LRRR

SEQ ID NO: 587 - protein, 4 aa, Artificial sequence
  Peptide fragment amino acid residues 217-220 of pHBP
NPPA

SEQ ID NO: 588 - protein, 221 aa, Sus sp.
IVGGRRAQPQEFPFLASIQKQGRPFCAGALVHPRFVLTAASCFRGKNSGSASVVLGAYDLRQQEQSRQTFSIRSISQNGYDPRQNLNDVLLLQLDREARLTPSVALVPLPPQNATVEAGTNCQVEAGWGTQRLRRLFSRFPRVLNVTVTSNPCLPRDMCIGVFSRRGRISQGDRGTPLVCNGLAQGVASFLRRRFRRSSGFFTRVALFRNWIDSVLNNPPA

SEQ ID NO: 589 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 590 - DNA, 678 bp, Homo sapiens
atcgttggcggccggaaggcgaggccccgccagttcccgttcctggcctccattcagaatcaaggcaggcacttctgcgggggtgccctgatccatgcccgcttcgtgatgaccgcggccagctgcttccaaagccagaaccccggggttagcaccgtggtgctgggtgcctatgacctgaggcggcgggagaggcagtcccgccagacgttttccatcagcagcatgagcgagaatggctacgacccccagcagaacctgaacgacctgatgctgcttcagctggaccgtgaggccaacctcaccagcagcgtgacgatactgccactgcctctgcagaacgccacggtggaagccggcaccagatgccaggtggccggctgggggagccagcgcagtggggggcgtctctcccgttttcccaggttcgtcaacgtgactgtgacccccgaggaccagtgtcgccccaacaacgtgtgcaccggtgtgctcacccgccgcggtggcatctgcaatggggacgggggcacccccctcgtctgcgagggcctggcccacggcgtggcctccttttccctggggccctgtggccgaggccctgacttcttcacccgagtggcgctcttccgagactggatcgatggcgttttaaacaatccgggaccggggccagcctag

SEQ ID NO: 591 - DNA, 660 bp, Sus sp.
attgtgggcggcaggagggcccagccgcaggagttcccgtttctggcctccattcagaaacaagggaggcccttttgcgccggagccctggtccatccccgcttcgtcctgacagcggccagctgcttccgtggcaagaacagcggaagtgcctctgtggtgctgggggcctatgacctgaggcagcaggagcagtcccggcagcattctccatcaggagcatcagccagaacggctatgayccccggcagaatctgaacgatgtgctgctgctgcagctggaccgtgaggccgactcacccccagtgtggccctggtaccgctgcccccgcagaatgccacagtggaagctggcaccaactgccaagttgcgggctgggggacccagcggcttaggaggcttttctcccgcttcccaagggtgctcaatgtcaccgtgacctcaaacccgtgtctccccagagcatgtgcattggtgtcttcagccgccggggccgcatcagccagggagacagaggcacccccctcgtctgcaacggcctggcgcagggcgtggcctccttcctccggaggcgtttccgcaggagctccggcttcttcacccgcgtggcgctcttcagaaattggattgattcagttctcaacaacccgccggcctga

SEQ ID NO: 592 - DNA, 750 bp, Homo sapiens
ctcgagaaaagaattgtgggtggccgtcgtgcccgtcctcacgcttggccgtttatggtgtctctgcagctgcgtggtggccacttctgcggtgcaaccctgattgcaccaaacttcgtcatgtccgcggcacactgcgtagcaaacgttaacgttcgtgcggtgcgtgtggttctgggtgctcataacctgtctcgtcgagaaccgacccgtcaagtgttcgccgtgcagcgcatcttcgaaaacggctacgacccggttaacctgctgaacgacatcgtgattctgcaactgaacggatccgccaccatcaacgccaacgtgcaagtggcacaactgccagcccaaggtcgccgcctgggaaacggagtacaatgcctggctatgggttggggcctgctcggccgtaaccgtggtatcgctagcgttctgcaagaactgaacgtgaccgtggttacctccctgtgtcgacgctctaacgtatgcactctggtgcgcggccgccaggctggcgtttgtttcggtgactccggtagcccgctggtttgcaacggtctgatccatggtattgcctccttcgttcgtggtggttgcgcctctggcctgtacccggatgcatttgccccggtggcacagtttgttaactggatcgactctatcattcagagatccgaagacaacccgtgtccgcacccacgtgatccagatccggcctccagaacacaccatcaccatcaccattaggaattc

SEQ ID NO: 593 - protein, 25 aa, Artificial sequence
  Peptide fragment amino acid residues 20-44 of pHBP
KQGRPFCAGALVHPRFVLTAASCFR

SEQ ID NO: 594 - protein, 78 aa, Artificial sequence
  Peptide fragment amino acid residues 96-173 of pHBP
REARLTPSVALVPLPPQNATVEAGTNCQVAGWGTQRLRRLFSRFPRVLNVTVTSNPCLPRDMCIGVFSRRGRISQGDR

SEQ ID NO: 595 - protein, 25 aa, Artificial sequence
  Peptide fragment amino acid residues 20-44 of hHBP with mutations N20K,Q44R
KQGRHFCGGALIHARFVMTAASCFR

SEQ ID NO: 596 - protein, 25 aa, Artificial sequence
  Peptide fragment amino acid residues 20-44 of  hHBP with mutations N2OK,H24P,Q44R
KQGRPFCGGALIHARFVMTAASCFR

SEQ ID NO: 597 - protein, 25 aa, Artificial sequence
  Peptide fragment amino acid residues 20-44 of hHBP with mutations N2OK,A33P,Q44R
KQGRHFCGGALIHPRFVMTAASCFR

SEQ ID NO: 598 - protein, 25 aa, Artificial sequence
  Peptide fragment amino acid residues 20-44 of hHBP with mutations N2OK,H24P,A33P,Q44R
KQGRPFCGGALIHPRFVMTAASCFR

SEQ ID NO: 599 - protein, 25 aa, Artificial sequence
  Reversed sequence amino acid residues 20-44 of pHBP.
RFCSAATLVFRPHVLAGACFPRGQK

SEQ ID NO: 600 - protein, 25 aa, Artificial sequence
  Peptide fragment amino acid residues 20-44 of pHBP with mutation K20N
NQGRPFCAGALVHPRFVLTAASCFR

SEQ ID NO: 601 - protein, 25 aa, Artificial sequence
  Peptide fragment amino acid residues 20-44 of pHBP with mutation R44Q
KQGRPFCAGALVHPRFVLTAASCFQ

SEQ ID NO: 602 - protein, 25 aa, Artificial sequence
  Peptide fragment amino acid residues 20-44 of pHBP with mutations  K20N,R44Q
NQGRPFCAGALVHPRFVLTAASCFQ

SEQ ID NO: 603 - protein, 25 aa, Artificial sequence
  Peptide fragment amino acid residues 20-44 of pHBP with mutation  R34Q
KQGRPFCAGALVHPQFVLTAASCFR

SEQ ID NO: 604 - protein, 25 aa, Artificial sequence
  Peptide fragment consisting of amino acid residues 20-44 of human neutrophil elastase
LRGGHFCGATLIAPNFVMSAAHCVA

SEQ ID NO: 605 - protein, 25 aa, Artificial sequence
  Peptide fragment consisting of amino acid residues 20-44 of murine neutrophil elastase
RRGGHFCGATLIARNFVMSAVHCVN

SEQ ID NO: 606 - protein, 25 aa, Artificial sequence
  Peptide fragment amino acid residues 20-44 of A. gambiae EAA01962
RSREYRCGGTLVSQRYILTAASCAA

SEQ ID NO: 607 - protein, 5 aa, Artificial sequence
  R1 amino acid sequence (claim 2)
KQGRP

SEQ ID NO: 608 - protein, 5 aa, Artificial sequence
  R1 amino acid sequence (claim 2)
KQGKP

SEQ ID NO: 609 - protein, 5 aa, Artificial sequence
  R1 amino acid sequence (claim 2)
RQGRP

SEQ ID NO: 610 - protein, 5 aa, Artificial sequence
  R1 amino acid sequence (claim 2)
RQGKP

SEQ ID NO: 611 - protein, 5 aa, Artificial sequence
  R1 amino acid sequence (claim 2)
NQGRH

SEQ ID NO: 612 - protein, 5 aa, Artificial sequence
  R1 amino acid sequence (claim 2)
NQGKH

SEQ ID NO: 613 - protein, 5 aa, Artificial sequence
  R19 amino acid sequences (claim 21)
PRGQK

(19) United States
(12) Patent Application Publication
(10) Pub. No.: US 2006/0173162 A1
(43) Pub. Date: Aug. 3, 2006

(54) Bactericidak anti-apoptotic, pro-inflammatory and anti-inflammatory peptides of heparin-binding protein (hbp)

(76) Inventors: Rene Djurup, Gentofte (DK); Hans Jakob Flodgaard, Hellerup (DK); Kjeld Norris, Charlottenlund (DK)

Correspondence Address: BROWDY AND NEIMARK, P.L.L.C.;624 NINTH STREET, NW, SUITE 300, WASHINGTON, DC, 20001-5303, US

(21) Appl. No.: 10/524,434

(22) PCT Filed: Aug. 14, 2003
(86) PCT No.: PCT/DK03/00542

§ 371 Date: Feb. 15, 2005

Related U.S. Application Data
(60) Provisional application No. 60/404,155, filed on Aug. 19, 2002.

(30) Foreign Application Priority Data
Aug. 15, 2002 (DK) PA 2002 01212
Jun. 27, 2003 (DK) PA 2003 00987

(51) Int. Cl. A61K 38/16; C07K 14/195

(52) U.S. Cl. 530/324; 514/12; 435/69.1; 435/320.1; 435/252.3; 536/23.7

(57) ABSTRACT

The present invention relates to providing new peptide fragments derived from the sequence of heparin-binding protein (HBP) and/or human neutrophil elastase and using said fragments for the manufacture of a medicament for the treatment of Gram positive and/or Gram negative infections. Thus, the peptides of the inventions can be used for treatment of pneumonia and/meningitis. The peptides of the invention may also be advantageously used for the treatment of any pathological condition demanding modulating of inflammatory response and/or a condition involving sepsis and/or, disseminated intravascular coagulation. Among other pathological conditions, which may be regarded for the treatment with the peptides of the invention, are conditions involving cell apoptosis.

### TECHNICAL FIELD OF THE INVENTION

The present invention relates to providing peptides derived from the sequence of heparin-binding protein (HBP) and/or human neutrophil elastase and using said peptides for the manufacture of a medicament for the treatment of Gram positive and/or Gram negative infections, sepsis, disseminated intravascular coagulation, modulation of inflammatory response, and/or prevention of cell apoptosis.

### BACKGROUND OF THE INVENTION

A local infection or injury in any tissue rapidly attracts white blood cells into the affected region as part of the inflammatory response, which helps fight the infection or heal the wound. The inflammatory response is complex and is mediated by a variety of signalling molecules produced locally by different types of cells. Some of these molecules act on nearby capillaries, causing the endothelial cells to adhere less tightly to one another but making their surfaces adhesive to passing white blood cells. Other molecules act as chemoattractants for specific types of blood cells, such as monocytes, causing these cells to become polarised and crawl toward the source of the attractant.

White blood cells, specifically polymorphonuclear leukocytes (PMNs), produce a large variety of peptides involved in the inflammatory response. Among these peptides is the heparin-binding protein (HBP), which was first isolated from azurophile granules of human PMNs. A highly homologous peptide was also isolated from PMNs of porcine origin and has been named porcine heparin-binding. protein (pHBP) (Flodgaard et al., 1991, Eur. J. Biochem. 197: 535-547; Pohl et al., 1990, FEBS Lett. 272: 200 ff.) HBP has otherwise been termed CAP37 (WO 91/00907, U.S. Pat. Nos. 5,458,874 and 5,484,885) and azurocidin (Wilde et al. 1990, J. Biol. Chem. 265:2038-41).

Sequence analysis of HBP has revealed that the protein bears many similarities to serine proteases, which are important in inflammatory processes, e. g. neutrophil elastase (47% homology) or protease 3 (43% homology), however HBP lacks protease activity due to mutations of two of three amino acids in the highly conserved catalytic triad. The structure of HBP appears from WO 89/08666 and Flodgaard et al., 1991 (Eur. J. Biochem. 197: 535-547).

HBP was originally studied because of its antibiotic and lipopolysaccharide binding properties (Gabay et al., 1989, Proc. Natl. Acad. Sci. U.S.A. 86:5610-5614 and Pereira et al., 1993, Proc. Natl. Acad. Sci. USA 90: 4733-7). However, a number of experimental evidence now supports the concept that HBP is a multifunctional protein, and, in addition to its bactericidal role, is involved during the progression of inflammation due to its effect on the recruitment and activation of monocytes (Pereira et al., 1990, J. Clin. Invest. 85:1468-1476, and Rasmussen et al., 1996, FEBS Lett. 390:109-112), recruitment of T cells (Chertov et al., 1996, J. Biol. Chem. 271:2935-2940), as well as on the induced contraction of endothelial cells and fibroblasts (Ostergaard and Flodgaard, 1992, J. Leuk. Biol. 51:316-323). Ostergaard and Flodgaard (op. cit.) also disclose increased survival of monocytes treated with HBP. Furthermore, in animal models of fecal peritonitis, HBP treatment has been shown to rescue mice from an otherwise lethal injury (Mercer-Jones et al., 1996, In: Surgical Forum, pp. 105-108; Wickel et al., 1997, In: 4th International Congress on the Immune Consequences of Trauma, Chock and Sepsis, Munich, Germany, pp. 413-416).

Using synthetic peptides derived from the sequence of human HBP in laboratory and preclinical research some functions of the protein has been structurally localised within the molecule of HBP. Thus, it has been shown that, for example, a high Gram negative bactericidal activity of human HBP is most probably associated with residues 20-44 of the human HBP amino acid sequence (Pereira et al., 1993, Proc. Natl. Acad. Sci. USA 90: 4733-7 and U.S. Pat. No. 6,107,460). The amino acid residues 95-122 of the human HBP sequence have been associated with a capacity of the protein to stimulate protein kinase C in vascular endothelial cells (Pereira et al., 1996, J. Leukoc. Biol. 60:415-22).

It would be advantageous to produce new peptides derived from the sequence of human HBP, porcine HBP, or analogues of these sequences, such as, for example, neutrophil elastase, to use for the manufacture of new bactericidal, anti-apoptotic medicaments and medicaments for modulation of an inflammatory response, especially the inflammatory response to bacterial infection.

### SUMMARY OF THE INVENTION

Thus, in one embodiment the present invention relates to providing peptides having a peptide having a sequence of at most 44 amino acid residues comprising a motif of the formula
X¹-X²-Cys-X³-X⁴-X⁵-X⁶-X⁷-X⁸-X⁹-X¹⁰-X¹¹-X¹²-X¹³-X¹⁴-X¹⁵-X¹⁶-X¹⁷-Cys-X¹⁸-X¹⁹,
wherein
the side chains of the two Cys residues are connected via a disulfide bond,
wherein
X can be an amino acid sequence or a single amino acid residue selected either from Group 1 consisting of Ala, Gly, and Ser,
Group 2 consisting of Arg and Lys,
Group 3 consisting of His, Ile, Leu, Met, Phe, Pro, Thr, Val, Trp, and Tyr,
Group 4 consisting of Asn and Gln, or
Group 5 consisting of Ala, Asn, Arg, Gln, Gly, His, Ile, Leu, Lys, Met, Phe, Pro, Ser, Thr, Trp, Tyr, Val,
wherein
X¹ can be represented by a sequence consisting of 2-5 amino add residues or an amino acid residue selected from Group 2;
X² is selected from Group 5 or Group 3;
X³, X¹⁵ and X⁴ are selected from Group 1;
X⁵ is Thr or selected from from Group 1;
X⁶, X¹¹, X¹², X¹³ and X⁷ are selected from Group 3;
X⁸ and X¹⁷ are selected from Group 1, 3 or 4;
X⁹ is selected from from Group 5, 1 or 3;
X¹⁰ is selected from from Group 2, 3 or 4;
X¹⁴ is Ser or selected from from Group 3;
X¹⁶ and X¹⁸ is selected from Group 1 or 3
X¹⁹ can be represented by a sequence consisting of 2-5 amino acid residues or a single amino acid residue selected from Group 5, 2, or 4,
with the proviso, that when X¹ includes Pro, then X¹⁹ is Gln.

The invention concerns providing peptides derived from the sequence of human HBP (hHBP) and/or porcine HBP (pHBP) and/or human neutrophil elastase capable of proinflammatory or anti-inflammatory activity, bactericidal and/or monocyte attractive activity, and/or capable of preventing cell apoptosis.

Further, the present invention discloses a recombinant process for the production of the above peptides, and the use of the peptides of the invention for the manufacture of a medicament for prevention or treatment of Gram negative and/or Gram positive bacterial infections, sepsis, severe sepsis, septic shock, disseminated and/or intravascular coagulation, stimulation or inhibition of the inflammatory response, or cell apoptosis.

### FIGURES

FIG. 1 depicts IL-6 secretion induced by HBP peptides in the absence of bacterial components.

FIG. 2 shows the effect of HBP 20-44 peptides on LPS induced IL-6 secretion

FIG. 3 shows the effect of HBP 20-44 peptides on LPS induced IL-6 secretion in the presence of PGN.

FIG. 4 shows the effect of HBP 20-44 peptides on LPS induced IL-6 secretion in the presence of PCW.

FIG. 5 shows the effect of N-Ac, C-amido hHBP 20-44 on PGN induced IL-6 secretion.

FIG. 6 shows the effect of R34Q pHBP 20-44 on PGN induced IL-6 secretion.

FIG. 7 shows the effect of different substitutions in the sequence of hHBP and p HBP on production of IL-6 induced by LPS.

FIG. 8 shows the effect of different substitutions in the sequence of hHBP and p HBP on production of IL6 induced by PGN.

Table 1 shows the potential applications for mono-functional peptides of the invention.

### DETAILED DESCRIPTION OF THE INVENTION

### Inflammation

The present invention relates to providing peptides and using said peptides for the manufacture of a medicament for modulation of the inflammatory response.

Inflammation is a defence reaction caused by tissue damage due to a mechanical injury or bacterial, virus or other organism infection. The inflammatory response involves three major stages: first, dilation of capillaries to increase blood flow; second, microvascular structural changes and escape of plasma proteins from the bloodstream; and third, leukocyte transmigration through endothelium and accumulation at the site of injury and infection. The inflammatory response begins with a release of inflammatory mediators. Inflammatory mediators are soluble, diffusible molecules that act locally at the site of tissue damage and infection, and at more distant sites, influencing consequent events of the inflammatory response. Inflammatory mediators can be exogenous, e. g. bacterial products or toxins, or endogenous, which are produced within the immune system itself, as well as injured tissue cells, lymphocytes, mast cells and blood proteins.

In one aspect the present invention relates to the inflammatory response to bacterial infection.

By “bacterial infection” in the present context is meant the invasion of normally sterile host tissue by bacteria. Bacterial infection of the invention may be due to invasion of either Gram negative or Gram positive bacteria, or a combination thereof or other infectious agents including fungi and virus. In one embodiment the present invention relates to the inflammatory response due invasion of Gram negative bacteria selected from the group comprising Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, Chromobacterium

In another embodiment the invention relates to the inflammatory response due to invasion by Gram positive bacteria selected from the group comprising Bacillaceae, Micrococcaceae (for example Staphylococcus aureus), Mycobacteriaceae (for example Staphylococcus pneumoniae), Peptococcaceae.

In an additional another embodiment the invention relates to the inflammatory response associated with sepsis, severe sepsis and/or septic shock.

By “sepsis” in the present context is meant the systematic inflammatory response to bacterial infection, characterised by one or more of the following conditions as a result of infection: temperature>38° C. or <36° C., heart rate>90 beats/min, respiratory rate>20 breaths/min or PaCO₂<32 torr (<4.3 kPa), and WBC>12 000 cells/mm³ or <4000 cells/mm³ or 10% immature (band) forms.

By “severe sepsis” in the present context is meant sepsis associated with organ dysfunction, hypoperfusion, or hypotension, hypoperfusion and hypotension abnormalities may include, but are not limited to, lactic acidosis (acidic condition in blood), oliguria (meaning reduction in urine production), or acute alteration in mental status.

By “septic shock” in the present context is meant sepsis with hypotension despite adequate fluid resuscitation, along with the presence of perfusion abnormalities that may include, but are not limited to, lactic acidosis, oliguria, or acute alteration in mental status.

In yet another embodiment the invention relates to the inflammatory response associated with disseminated intravascular coagulation (DIC).

By “DIC” in the present context is meant a pathophysiologic condition involving a continuum of events that occur in the coagulation pathway in association with a variety of well-defined clinical situations, including sepsis, major trauma, and abruptio placenta, and with laboratory evidence of the following: procoagulant activation, fibrinolytic activation, inhibitor consumption and biochemical evidence of end-organ damage or failure.

Proinflammatory Peptides

It is an objective of the present invention to provide new peptides, which are capable to serve as additional mediators of the inflammatory response, the so-called proinflammatory peptides are particularly useful but not limited to patients selected from groups of immune-suppressed patients, cancer patients, patients with autoimmune diseases and patients undergoing major surgery.

In the present context by the term “pro-inflammatory peptide” is meant an artificial peptide compound which is capable of
i) Stimulating, either alone or in synergistic action with bacterial products including, but not limited to LPS (Lipopolysaccharide), PGN (peptidoglycan), LTA (Lipotechoic acid), MDP (muramyldipeptide) and PCW (purified cell wall from bacteria), the gene expression in the immune cells, preferably monocytes/macrophages, leading to secretion of endogenous inflammatory mediators including receptors for inflammatory mediators and transcription factors involved in the signal tranduction of the inflammatory mediators, said mediators being preferably selected from the group comprising cytokines, selected from the group TNFalpha IL-1, IL-6, G-CSF, GM-CSF, M-CSF. Chemokines selected from the group comprising IL-8, MCP-1, receptors selected from the group Tissue factor and IL-2Ralpha. and/or
ii) activating the production of bradykinin by the phase contact system, and/or;
iii) serving as an attractant for monocytes, and/or
iv) increasing the life-time of monocytes, neutrophils and other immune cells serving as an inhibitor of apoptosis, and/or
v) activating vascular endothelial cells to express the adhesion molecules, said adhesion molecules being preferably selected from the group comprising PECAM, ICAM-1, E-selectins, VCAM-1, and/or
vi) activate the contact phase system to produce bradykinin leading to an increased vascular permeability, and/or
vii) increase the phagocytic potential of monocytes/macrophages, and/or
viii) upregulate class-II MHC.

In one embodiment the pro-inflammatory peptide of the invention is a peptide
i) derived from any of the sequences set forth in SEQ ID NO: 1, SEQ ID NO:588, or SEQ ID NO: 589,
ii) comprising one or more of the sequences set forth in SEQ ID NO: 2-587,
iii) capable of at least one of the above activities (i-viii) of an pro-inflammatory compound, more preferable at least two of the above activities, even more preferable at least tree of the above activities, even more preferable at least four of the above activities, even more preferable at least five of the above activities, even more preferable at least six of the above activities, even more preferable at least seven of the above activities, and most preferably eight of the above activities.

In the present context the term “synergistic action” refers to the situation where the combined action of a bacterial product and a peptide of the present invention is a stronger pro-inflammatory stimulant than the pro-inflammatory stimulant a bacterial product or the present peptide, respectively would be on their own.

In another embodiment the invention provides a pro-inflammatory peptide capable of stimulating either alone or in synergistic action with bacterial products including, but not limited to LPS (Lipopolysaccharide), PGN (peptidoglycan), LTA (Lipotechoic acid), MDP (muramyldipeptide) and PCW (purified cell wall from bacteria) the secretion of cytokine IL-6 from monocytes, comprising two or more sequences set forth in SEQ ID NOS: 15-36, wherein said sequences constitute a contiguous sequence derived from the sequence of hHBP set forth in SEQ ID NO:1. Further, the pro-inflammatory peptide may be used for the manufacture of a medicament for the treatment of individuals having suppressed immune system, cancer, autoimmune diseases and/or trauma.

In a preferred embodiment the invention concerns an pro-inflammatory peptide, wherein said peptide has the sequence NQGRHFCGGALIHARFVMTAASCFQ (SEQ ID NO: 594). Even more preferred the peptide, wherein the sequence identified in SEQ ID NO: 594 has the N-terminal and C-terminal modified, as for example the N-terminal amino group being amidated and the C-terminal carboxy group being acetylated.

Anti-Inflammatory Peptides

It is another important objective of the invention to provide new anti-inflammatory peptide, which are capable of serving as inhibitors of the sustained inflammatory response.

The continuous presence of inflammatory mediators, such as for example TNF alpha in the body in response to sustained presence of bacterial products or even live bacteria locally during days or weeks following trauma and/or infection promotes the reactions to inflammation, such as, for example, heat, swelling, and pain. The sustained inflammatory response has been proven to be very harmful to the body. If the bacterial products or live bacteria become spread universally in the body from their local focus the inflammatory reaction becomes overwhelming and out of control and leads to sepsis which eventually progress further to severe sepsis and septic shock. Anti-inflammatory peptides may be used to block or suppress the overwhelming sustained inflammatory response represented by a massive and harmful cytokine cascade in the blood and vital organs such as lung, liver intestine, brain and kidneys.

In the present context by the term “anti-inflammatory compound” is meant a compound which is capable of
i) decreasing or inhibiting the gene expression in the immune cells, preferably monocytes/macrophages in response to bacterial products, live bacteria or trauma to produce endogenous inflammatory mediators including receptors for inflammatory mediators and transcription factors involved in the signal transduction of the inflammatory mediators, said mediators being preferably selected from the group comprising cytokines, selected from the group TNFalpha IL-1, IL-6, G-CSF, GM-CSF, M-CSF. Chemokines selected from the group comprising IL8, MCP-1, receptors selected from the group Tissue factor and IL-2Ralpha. and/or
ii) decrease or inhibit the production bradykinin by the phase contact system, and/or;
iii) decrease or inhibit the attractant potential for monocytes, and/or
iv) decrease or inhibit the life-time of monocytes, neutrophils and other immune cells serving as an inducer of apoptosis, and/or
v) decrease or inhibit vascular endothelial cells to express the adhesion molecules, said adhesion molecules being preferably selected from the group comprising PECAM, ICAM-1, E-selectins, VCAM-1 and/or
vi) decrease or inhibit activation of the contact phase system to produce bradykinin leading to increased vascular permeability, and/or
vii) stimulate the synthesis of an anti-inflammatory mediator selected from the group of IL-10 and IL-12, and/or
viii) removing endotoxin from septic patients, and/or

In one embodiment the anti-inflammatory peptide compound of the invention is a peptide
i) derived from any of the sequences set forth in SEQ ID NO: 1, SEQ ID NO: 588, or SEQ ID NO: 589,
ii) comprising one or more of the sequences set forth in SEQ ID NO: 2-587,
iii) capable of at least one of the above activities of an anti-inflammatory compound, more preferable at least two of the above activities, even more preferable at least tree of the above activities, even more preferable at least four of the above activities, even more preferable at least five of the above activities, even more preferable at least six of the above activities, even more preferable at least seven of the above activities, even more preferable at least eight of the above activities, and most preferably nine of the above activities.

In another embodiment the invention provides an anti-inflammatory peptide capable of inhibiting the secretion of cytokine IL-6 from monocytes in response to bacterial products including, but not limited to, LPS (Lipopolysaccharide), PGN (peptidoglycan), LTA (Lipotechoic acid), MDP (muramyldipeptide) and PCW (purified cell wall from bacteria), comprising two or more sequences set forth in SEQ ID NOS: 233-253, wherein said sequences constitute a contiguous sequence derived from the sequence of pHBP set forth in SEQ ID NO:588.

In a preferred embodiment of the invention an inflammatory peptide has the sequence KQGRPFCAGALVHPRFVLTAASCFR (SEQ ID NO: 593). Even more preferred the peptide, wherein the sequence identified in SEQ ID NO: 593 has the N-terminal and C-terminal modified, as for example the N-terminal amino group being amidated and the C-terminal carboxy group being acetylated.

### Peptides

It is an objective of the present invention to provide one or more peptides for the manufacture of a medicament for prevention and/or treatment of Gram positive and/or Gram negative infections, sepsis, severe sepsis, septic shock and/or disseminated intravascular coagulation, and/or for modulation of inflammatory response, and/or prevention of cell apoptosis.

In one embodiment the invention concerns a peptide having a sequence of at most 44 amino acid residues comprising a motif of the formula
X¹-X²-Cys-X³-X⁴-X⁵-X⁶-X⁷-X⁸-X⁹-X¹⁰-X¹¹-X¹²-X¹³-X¹⁴-X¹⁵-X¹⁶-X¹⁷-Cys-X¹⁸-X¹⁹,
wherein
the side chains of the two Cys residues are connected via a disulfide bond,
wherein
X can be an amino acid sequence or a single amino acid residue selected either from Group 1 consisting of Ala, Gly, and Ser,
Group 2 consisting of Arg and Lys,
Group 3 consisting of His, Ile, Leu, Met, Phe, Pro, Thr, Val, Trp, and Tyr,
Group 4 consisting of Asn and Gln, or
Group 5 consisting of Ala, Asn, Arg, Gln, Gly, His, Ile, Leu, Lys, Met, Phe, Pro, Ser, Thr, Trp, Tyr, Val,
wherein
X¹ can be represented by a sequence consisting of 2-5 amino acid residues or an amino acid residue selected from Group 2;
X² is selected from Group 5 or Group 3;
X³, X¹⁵ and X⁴ are selected from Group 1;
X⁵ is Thr or selected from from Group 1;
X⁶, X¹¹, X¹², X¹³ and X⁷ are selected from Group 3;
X⁸ and X¹⁷ are selected from Group 1, 3 or 4;
X⁹ is selected from from Group 5, 1 or 3;
X¹⁰ is selected from from Group 2, 3 or 4;
X¹⁴ is Ser or selected from from Group 3;
X¹⁶ and X¹⁸ is selected from Group 1 or 3
X¹⁹ can be represented by a sequence consisting of 2-5 amino acid residues or a single amino acid residue selected from Group 5, 2, or 4,
with the proviso, that when X¹ includes Pro, then X¹⁹ is Gln.

In a preferred embodiment X¹ in sequence of the peptide of the invention is represented by an amino acid sequence selected from SEQ ID NOS: 607-612. In another preferred embodiment X¹ is Arg. Another preferred embodiment for the sequence of the peptide is Phe in position X². It is also preferred Ala or Gly as the X³ residue. The peptide preferably has Gly as the X⁴ residue. The X⁵ residue is preferably represented by Ala, and X⁶ by Leu. The X⁷ may preferably be selected from the group containing Ile, Leu, Met or Val. The peptide according to another preferred embodiment has X⁸ represented by His or Val. The position X⁹ in the sequence of the peptide may preferably be occupied by a residue selected from Ala, Phe or Pro. X¹⁰ is preferably represented by Arg, and X¹¹ is Phe or Pro. The position X¹² is preferably occupied by His or Val, and X¹³ is preferably selected from the group consisting Ile, Leu, Met or Val. The peptide has preferably Thr in position X¹⁴, Ala in position X¹⁵ and X¹⁶, and X¹⁷ is preferably Ser. The X¹⁸ residue is preferably represented by Phe. X¹⁹ in one preferred embodiment is represented by a sequence identified as SEQ ID NO: 613, and in another preferred embodiment is represented by Arg or Gln.

Another preferred embodiment of a peptide having the sequence comprising the above motif is that N-terminal and/or C-terminal of said peptide may be modified. Thus, the C-terminal carboxy group in a more preferred embodiment is amidated, and the N-terminal in another more preferred embodiment is acetylated.

Furthermore, the invention in the other preferred embodiments concerns particular amino acid sequences comprising the motif disclosed above. Thus, the peptide according to the present invention preferably has a sequence selected from

| | |
|---|---|
| KQGRPFCAGALVHPRFVLTAASCFR, | (SEQ ID NO: 593) |
| NQGRHFCGGALIHARFVMTAASCFQ, | (SEQ ID NO: 594) |
| KQGRHFCGGALIHARFVMTAASCFR, | (SEQ ID NO: 595) |
| KQGRPFCGGALIHARFVMTAASCFR, | (SEQ ID NO: 596) |
| KQGRHFCGGALIHPRFVMTAASCFR, | (SEQ ID NO: 597) |
| KQGRPFCGGALIHPRFVMTAASCFR, | (SEQ ID NO: 598) |
| RFCSAATLVFRPHVLAGACFPRGQK, | (SEQ ID NO: 599) |
| NQGRPFCAGALVHPRFVLTAASCFR, | (SEQ ID NO: 600) |
| KQGRPFCAGALVHPRFVLTAASCFQ, | (SEQ ID NO: 601) |
| NQGRPFCAGALVHPRFVLTAASCFQ, | (SEQ ID NO: 602) |
| KQGRPFCAGALVHPQFVLTAASCFR, | (SEQ ID NO: 603) |
| LRGGHFCGATLIAPNFVMSAAHCVA, | (SEQ ID NO: 604) |
| RRGGHFCGATLIARNFVMSAVHCVN | (SEQ ID NO: 605) |
| and | |
| RSREYRCGGTLVSQRYILTAASCAA. | (SEQ ID NO: 606) |

The sequences identified in SEQ ID NO: 593 and 594 according to the invention are preferably modified in the N- and C-terminus as described above.

All peptides of at most 44 amino acids length comprising the motif as above are in the scope of the present invention. However, the invention preferably concerns peptides wherein the positions X¹⁵-X¹⁶-X¹⁷ in the motif are Ala-Ala-Ser correspondingly, in positions X¹ to X¹⁴ and X¹⁸ to X¹⁹ may be any amino acid residues connected in a contiguous polypeptide chain. The preferred embodiments for the positions are as described above. Moreover, it is further preferred, if the peptide comprises one or more amino acid sequences are selected from SEQ ID NOS: 12-39. Another preferred embodiment if the sequence comprises one or more amino acid sequences are selected from SEQ ID NOS: 233-253. It is still another preferred embodiment if the peptide comprises one or more amino acid sequences are selected from SEQ ID NOS: 233-253and SEQ ID NO: 34. In yet preferred embodiment the peptide comprises one or more amino acid sequences selected from SEQ ID NOS: 233-253 and SEQ ID NO: 21. Still yet preferred embodiment is a peptide comprising one or more amino acid sequences are selected from SEQ ID NOS: 233-253 and SEQ ID NO: 21 and SEQ ID NO: 34. Also preferred a peptide comprising one or more amino acid sequences are selected from SEQ ID NOS: 395-421.

The sequences of the peptide may be derived from any longer polypeptide sequence of natural or artificial origin. However the invention concerns some preferred embodiments for such the sequence. In a first preferred embodiment the invention concerns one or more amino acid sequences which are derived from the sequence of human heparin-binding protein (hHBP) set forth in SEQ ID NO: 1. In another preferred embodiment the invention concerns one or more amino acid sequences derived from the sequence of porcine heparin-binding protein (pHBP) set forth in SEQ ID NO: 588. In still another preferred embodiment the invention concerns one or more amino acid sequences derived from the sequence of human neutrophil elastase set forth in SEQ ID NO: 589.

Additionally, a preferred peptide comprising the above motive according to the invention comprises one or more amino acid sequences set forth in SEQ ID NOS: 2-587.

A peptide as described above according to the invention is capable of inhibiting the secretion of cytokine IL-6 from monocytes.

It is another objective of the present invention to produce peptides as small as possible, yet exhibiting the desired effect(s).

In one embodiment, the invention relates to providing a peptide consisting of at most 8 amino acids comprising one or more of the amino acid sequences set forth in SEQ ID NOS: 2-587.

In another embodiment, the invention relates to providing a peptide consisting of at most 12 amino acids comprising one or more of the amino acid sequences set forth in SEQ ID NOS: 2-14, 22-36, 46-107, 115-185 and 195-587.

In still another embodiment, the invention relates to providing a peptide consisting of at most 16 amino acids comprising one or more of the amino acid sequences set forth in SEQ ID NOS: 2-14, 46-107, 115-185 and 195-587.

In yet another embodiment, the invention relates to providing a peptide consisting of at most 20 amino acids comprising one or more of the amino acid sequences set forth in SEQ ID NOS: 46-107, 115-185 and 195-587.

In yet still another embodiment, the invention relates to providing a peptide consisting of at most 24 amino acids comprising one or more of the amino acid sequences set forth in SEQ ID NOS: 46-89, 117-124, 139-157, 163-175 and 195-587.

In yet another embodiment, the invention relates to providing a peptide consisting of at most 28 amino acids comprising one or more of the amino acid sequences set forth in SEQ ID NOS: 46-52, 61-66, 73-89, 117-124, 165-175 and 195-587.

In still another embodiment, the invention relates to providing a peptide consisting of at most 32 amino adds comprising one or more of the amino add sequences set forth in SEQ ID NOS: 46-52, 61-66, 73-89, 117-124, 165-175 and 195-587.

In still further another embodiment, the invention relates to providing a peptide consisting of at most 36 amino acids comprising one or more of the amino acid sequences set forth in SEQ ID NO: 46-52, 61-66, 73-89, 117-124, 165-175 and 195-587.

In yet another embodiment, the invention relates to providing a peptide consisting of at most 40 amino acids comprising one or more of the amino acid sequences set forth in SEQ ID NO: 46-52, 61-66, 73-89, 117-124, 165-175 and 195-587.

In yet still another embodiment, the invention relates to providing a peptide consisting of at most 44 amino acids comprising one or more of the amino acid sequences set forth in SEQ ID NO: 46-52, 61-66, 73-89, 117-124, 165-175 and 195-587.

Furthermore, in yet another embodiment, the invention relates to providing a peptide consisting of at least 48 amino acids and at most 224 amino acids comprising one or more of the amino acid sequences set forth in SEQ ID NOS: 2-587.

According to amino acid sequences of the above peptides may be derived from the amino acid sequence of polypeptides selected from the group comprising hHBP (SEQ ID NO: 1), pHBP (SEQ ID NO: 588), or human neutrophil elastase (SEQ ID NO: 589).

In one embodiment the peptide comprises at least two of the sequences set forth above. In such embodiment it is preferred that the two or more sequences constitute a continuous sequence derived from another sequence, such as a continuous sequence derived from hHBP, or pHBP, or human neutrophil elastase.

In another embodiment the peptide comprises at least two of the sequences set forth above. In such embodiment it is preferred that the two or more sequences are randomly selected to constitute a continuous sequence derived from another sequence, such as a random sequence derived from hHBP, or pHBP, or human neutrophil elastase.

In the present context by the term “derived from” is meant that one amino acid sequence, such as for example a peptide amino acid sequence, is representing a fragment, or is comprising a fragment of another amino acid sequence, such as for example the amino acid sequence of a larger polypeptide: thus, the peptide amino acid sequence is derived from (originates from) the amino acid sequence of the larger polypeptide.

In an additional embodiment the present invention relates to providing a peptide consisting of at least 24 amino acids and at most 224 amino acids comprising one or more of the amino acid sequences set forth in SEQ ID NOS: 233-253.

In another additional embodiment the present invention relates to providing a peptide consisting of at least 24 amino acids and at most 224 amino acids comprising one or more of the amino acid sequences set forth in SEQ ID NOS: 286-346.

In a preferred embodiment the present invention relates to providing a peptide having the sequence KQGRPFCAGALVHPRFVLTAASCFR set forth in SEQ ID NO: 593, or fragments of said sequence, or variants of said sequence, or fragments of said variants.

In another preferred embodiment the present invention relates to providing a peptide having the sequence NQGRHFCGGALIHARFVMTAASCFQ set forth in SEQ ID NO: 594, or fragments of said sequence, or variants of said sequence, or fragments of said variants.

By the term of “fragment” in the present context is meant that a peptide of the invention is represented by a shorter amino acid sequence which is identical to any of the amino acid sequences which the peptide comprises.

By the term “variant” in the present context is meant that a peptide of the invention is represented by an amino acid sequence which has at least 40% identity with the amino acid sequence of the peptide, more preferably at least 50%, even more preferably at least 60%, even more preferably at least 70%, even more preferably at least 80%, even more preferably at least 90%, even more preferably at least 95%, and most preferably at least 97%.

The amino acid sequence of a variant of a peptide may differ from the amino acid sequence of the peptide by an insertion or deletion of one or more amino acid residues and/or the substitution of one or more amino acid residues by different amino acid residues. Preferably, amino acid changes are of a minor nature, that is, conservative amino acid substitutions; small deletions, typically of one to about 10 amino acids; small amino- or carboxyl-terminal extensions; small linker sequences of about 3-15 residues; or a small extension that may facilitates purification by changing net charge or another function, such as a polyhistidine tract, an antigenic epitope or a binding domain.

Examples of conservative substitutions are within the group of basic amino acids (such as arginine, lysine and histidine), acidic amino acids (such as glutamic acid and aspartic acid), polar amino acids (such as glutamine and asparagine), hydrophobic amino acids (such as leucine, isoleucine and valine), aromatic amino acids (such as phenylalanine, tryptophan and tyrosine) and small amino acids (such as glycine, alanine, serine, threonine and methionine). Amino acid substitutions, which do not generally alter the specific activity, are known in the art and are described, e.g., by H. Neurath and R. L. Hill, 1979, in, The Proteins, Academic Press, New York. The most commonly occurring exchanges are: Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly as well as these in reverse.

It is an additional aspect of the present invention to provide functional fragments or variants of the peptides.

By the term “functional” in relation to a peptide fragment or peptide variant in the present context is meant that the peptide fragment or peptide variant is capable to demonstrate one or more of the biological activities described below.

In a preferred embodiment the invention relates to providing functional fragments, variants or fragments of said variants of a peptide having the sequence KQGRPFCAGALVHPRFVLTAASCFR (SEQ ID NO: 593).

In another preferred embodiment the invention relates to providing functional fragments, variants or fragments of said variants of a peptide having the sequence NQGRHFCGGALIHARFVMTAASCFQ (SEQ ID NO: 594).

It is an object of the invention to provide a peptide, wherein said peptide comprises one or more amino acid sequences set forth in SEQ ID NOS: 233-253 having the motif cys-X₁₅-cys, wherein X₁₅ represents an amino acid sequence of 15 amino acids.

In a further embodiment said peptide comprises one or more amino acid sequences set forth in SEQ ID NOS: 233-253 and the amino acid sequence set forth in SEQ ID NO: 34.

In yet a further embodiment the present peptide comprises one or more amino acid sequences set forth in SEQ ID NOS: 233-253and the amino acid sequence set forth in SEQ ID NO: 21.

In another aspect the peptide comprises one or more amino acid sequences set forth in SEQ ID NOS: 233-253 and the amino acid sequence set forth in SEQ ID NO: 34 and the amino acid sequence set forth in SEQ ID NO: 21.

Furthermore, it is within the scope of the invention to provide a peptide, which
i) comprises a sequence derived from any of the amino acid sequences set forth in SEQ ID NO: 1, SEQ ID NO: 588, or SEQ ID NO: 589, and
ii) is capable of bactericidal activity, and/or
iii) is an attractant for monocytes.

In one embodiment said peptide is consisting of at most 8 amino acids, whereof at least 5 and at most 6 amino acids are basic amino acids selected from the group comprising lysine, arginine and histidine.

In another embodiment the peptide is consisting of at most 12 amino acids, whereof at least 6 and at most 9 amino acids are basic amino acids selected from the group comprising lysine, arginine and histidine.

In still another embodiment the peptide is consisting of most 16 amino acids, whereof at least 8 and most 12 amino acids are basic amino acids selected from the group comprising lysine, arginine and histidine.

In still further another embodiment the peptide is consisting of at the most 20 amino acids, whereof at least 10 and at most 15 amino acids are basic amino acids selected from the group comprising lysine, arginine and histidine.

In still yet another embodiment the peptide is consisting of at most 24 amino acids, whereof at least 12 and at most 18 amino acids are basic amino acids selected from the group comprising lysine, arginine and histidine.

In yet another embodiment the peptide is consisting of at most 28 amino acids, whereof at least 14 and at most 21 amino acids are basic amino acids selected from the group comprising lysine, arginine and histidine.

In yet further another embodiment the peptide is consisting of at most 32 amino acids, whereof at least 16 and at most 24 amino acids are basic amino acids selected from the group comprising lysine, arginine and histidine.

In yet still further another embodiment the peptide is consisting of at most 36 amino acids, whereof at least 18 and at most 27 amino acids are basic amino acids selected from the group comprising lysine, arginine and histidine.

In a further another embodiment the peptide is consisting of at most 40 amino acids, whereof at least 20 and at most 30 amino acids are basic amino acids selected from the group comprising lysine, arginine and histidine.

Moreover, the invention also provides a peptide, which
i) comprises a sequence derived from any of the amino acid sequences set forth in SEQ ID NO: 1, SEQ ID NO: 588, or SEQ ID NO: 589, and
ii) is capable of preventing cell apoptosis.

In one embodiment said peptide is consisting of at most 8 amino acids, whereof at least 4 and at most 6 amino acids are acidic amino acids selected from the group comprising aspartic acid and glutamic acid.

In another embodiment the peptide in consisting of at most 12 amino acids, whereof at least 6 and at most 10 amino acids are acidic amino acids selected from the group comprising aspartic acid and glutamic acid.

In still another embodiment the peptide is consisting of at most 16 amino acids, whereof at least 8 and at most 12 amino acids are acidic amino acids selected from the group comprising aspartic acid and glutamic acid.

In yet another embodiment the peptide is consisting of at most 20 amino acids, whereof at least 10 and at most 12 amino acids are acidic amino acids selected from the group comprising aspartic acid and glutamic acid.

In still yet another embodiment the peptide is consisting of at most 24 amino acids, whereof at least 12 and at most 18 amino acids are acidic amino acids selected from the group comprising aspartic acid and glutamic acid.

In yet further another embodiment the peptide is consisting of at most 28 amino acids, whereof at least 14 and at most 21 amino acids are acidic amino acids selected from the group comprising aspartic acid and glutamic acid.

In yet still further another embodiment of at most 32 amino acids, whereof at least 16 and at most 24 amino acids are acidic amino acids selected from the group comprising aspartic acid and glutamic acid.

Furthermore, in yet still further another embodiment the peptide is consisting of at most 36 amino acids, whereof at least 18 and at most 27 amino acids are acidic amino acids selected from the group comprising aspartic acid and glutamic acid.

In a further another embodiment the peptide is consisting of at most 40 amino acids, whereof at least 20 and at most 30 amino acids are acidic amino acids selected from the group comprising aspartic acid and glutamic acid.

### Screening Assays

According to the invention recombinant or synthetically produced peptides are further screened for their biological activity.

In the present content by “biological activity of a peptide” is meant that a peptide is able to demonstrate at least one of the following biological activities: (1) heparin binding, (2) lipopolysaccaride (LPS) binding; (3) activating of protein kinase C; (4) stimulating thrombospondin secretion from monocytes; (5) stimulating/inhibiting the production of IL-1, IL-6, IL-8, GCSF, GM-CSF, M-CSF, TNF-α, MCP-1, group Tissue factor, IL-2R-α; (6) bactericidal; (7) chemotactic for monocytes; (8) anti-apoptotic, (9) stimulating/inhibiting the vascular permeability; (10) stimulating/inhibiting the expression of adhesion molecules PECAM or ICAM1 by endothelial cells, (11) stimulating/inhibiting the production of bradykinin, (12) increase the phagocytic potential, (13) up-regulate class-II MHC.

In a preferred embodiment the peptide is able to demonstrate at least two of the above activities, more preferably at least tree of the above activities, even more preferably at least four of the above activities (1-11), yet even more preferably at least five of the above activities, even more preferably at least six of the above activities, even more preferably at least seven of the above activities, even more preferably at least eight of the above activities, even more preferably at least nine of the above activities, even more preferably at least ten of the above activities and most preferably the peptide is able to demonstrate at least all of the above activities.

Methods for evaluating of the above listed biological activities of peptides according to the invention are well known in art.

According to the invention there are a number of available assays for evaluating the biological activity of the present peptide.

One of such assays for the evaluation of chemotactic activity of the peptides may for example be the method of Cates et al. (in Leukocyte chemotaxis, p 67. Gallin and Quie eds, Raven Press, NY, 1978), or of Keire et al. (J. Biol. Chem. 2001, 276: 48847-53).

In another embodiment the lipopolysaccharide-binding activity of the peptides may be examined by a method described by Linde et al (Biotechniques 2000, 28:218-20).

To evaluate the bactericidal activity of the present peptides, the assay described by Shafer et al. (Infect. Immun. 1986, 53:651-55) may be used.

In one aspect measuring cell apoptopsis in the presence of the present peptides may be done according to Linde et al. (Anal. Biochem. 2000, 280:186-8).

It is possible to perform an evaluation of the heparin binding capacity of the peptides by conventional chromatography on a commercially available heparin-affinity column.

The protein kinase C activation by the peptides may be done according to Pereira et al., 1996 (J. Leukoc. Biol. 60:415-22).

The changes in expression of different polypeptides, such as for example IL-1, IL-6, IL-8, TNF-α, thrombospondin, PECAM or ICAM in the presence of the peptides according to the invention may, for example, be evaluated either by reverse phase transcriptase, immunoassay, immunoblotting, or immunostaining of the treated cells grown in culture.

The vascular permeability may be determined by using the assay as described by Gautam et al. in 1998 (Br J Pharmacol 1998 November; 125(5):1109-14)

### Medicament

It is an important objective of the present invention to use the peptides, functionally active fragments or variants of said peptides, for the manufacture of a medicament for prevention and/or treatment of Gram positive and/or Gram negative infections, sepsis, severe sepsis, septic shock and/or disseminated intravascular coagulation, and/or for modulation of inflammatory response, and/or prevention of cell apoptosis.

In one embodiment the invention relates to the manufacture of a medicament which is capable of being used for prevention and/or treatment of Gram positive bacterial infection caused by Bacillaceae, Microccaceae, Mycobacteriaceae, Peptococcaceae and/or a Gram negative bacterial infection caused by Acetobacteriaceae, Alcaligenaceae, Bacteroidaceae, Chromatiaceae, Enterobacteriaceae, Legionelaceae, Neisseriaceae, Nitrobacteriaceae, Pseudomonadaceae, Rhizobiaceae, Rickettsiaceae, Spirochaetaceae, Vibrionaceae, Brucella, Chromobacterium.

In a preferred embodiment for prevention and/or treatment the infection by Neisserie meningitidis (meningococcus) and/or Pneumococcus pneumonae (pneumococcus).

In another embodiment the invention relates to the manufacture of a medicament which is capable of being used for prevention and/or treatment of sepsis, severe sepsis, septic shock and disseminated intravascular coagulation.

It is an important objective of the invention to use the peptides for the manufacture of a medicament for stimulation of an inflammatory response, in a preferred embodiment, the inflammatory response to bacterial infection.

Another important objective of the invention is to use the peptides for the manufacture of a medicament for inhibition of an inflammatory response. Examples of inflammatory responses, which may be harmful for an individual and therefore are advantageously being suppressed include but are not limited by conditions associated with extensive trauma, or chronic inflammation, such as for example type IV delayed hypersensitivity, associated for example with infection by Tubercle bacilli, or systematic inflammatory response syndrome, or multiple organ failure, or rheumatoid arthritis.

In an additional embodiment of the invention to use the peptides capable of anti-apoptotic activity are used for the manufacture of a medicament for the treatment of a disease, pathological conditions whereof are associated with massive cell loss due to apoptosis. Examples of such a disease include but not limited by degenerative diseases the central and peripheral nervous system, such as postoperative nerve damage, traumatic nerve damage, e.g. resulting from spinal cord injury, impaired myelination of nerve fibers, postischaemic damage, e.g. resulting from a stroke, multiinfarct dementia, multiple sclerosis, nerve degeneration associated with diabetes mellitus, neuro-muscular degeneration, schizophrenia, Alzheimer's disease, Parkinson's disease, or Huntington's disease, degenerative conditions of the gonads, of the pancreas, such as diabetes mellitus type I and II, of the kidney, such as nephrosis, or cancer.

By the term “apoptosis” in the present content is meant a programmed cell death due to activation an internal death program.

In the pharmaceutical composition of a medicament of the invention, the peptides and antibodies may be formulated by any of the established methods of formulating pharmaceutical compositions, e.g. as described in Remington's Pharmaceutical Sciences, 1985. The composition may typically be in a form suited for local or systemic injection or infusion and may, as such, be formulated with sterile water or an isotonic saline or glucose solution. The compositions may be sterilised by conventional sterilisaton techniques, which are well known in the art. The resulting aqueous solutions may be packaged for use or filtered under aseptic conditions and lyophilised, the lyophilised preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc. The concentration of peptides may vary widely, i.e. from less than about 0.5%, such as from 1%, to as much as 15-20% by weight. A unit dosage of the composition may typically contain from about 10 mg to about 1 g of a peptide.

The peptides and antibodies may be administered topically or by injection. Dosages will be prescribed by the physician according to the particular condition and the particular individual to be treated. Dosages and frequency is carefully adapted and adjusted according to parameters determined by the physician in charge. A preferred administration route may be e.g. subcutaneous injections. Subcutaneous, intravenous, intramuscular, intratracheal, intravesical, intratechal or intraperitoneal injections of HBP peptides and anti-HBP antibodies may be given per 24 hours in the range of from 0.1-100 mg, especially 0.1-20 mg, in particular 0.1-10 mg per kg body weight. The dose may be given 1-4 times per 24 hours or administered continuously through a catheter.

Compositions of a medicament used in the present invention comprising bioactive peptides of HBP, HBP homologous peptides or anti-HBP antibodies described below may additionally be supplemented by antibiotics, wherein said antibiotics are routinely prescribed antibiotics by the physician according to the particular condition and the particular individual to be treated. In a preferred embodiment the supplemented antibiotics are selected from but not limited by the group of beta-lactam antibiotics, comprising penicillins and cephalosporins. A medicament comprising a peptide of HBP or a fragment of a HBP homologous peptide or an anti-HBP antobody may still additionally be supplemented by an pro-inflammatory drug, or an anti-inflammatory drug, wherein said drugs are prescribed by the physician according to the particular condition and the particular individual to be treated. The supplementary pro-inflammatory drugs may for example be selected from the group comprising CSF (colony stimulating factor) drugs. The supplementary anti-inflammatory drugs may for example be selected from the group comprising antibiotics, steroids, cytostatics, or antiviral drugs.

HBP Receptors and Binding Sites

According to the invention information concerning potential HBP receptors and binding sites is aiding the selection of the present peptides. There has been no identification of a HBP receptor, but receptor-like structures or binding sites of HBP have been identified. HBP is a dipole separated by a hydrophobic cleft and it is therefore capable to interact with both positively and negatively charge surfaces and molecules and with hydrophobic molecules and epitomes. The charged surface areas (the epitomes) of HBP are important for several of its functions. Without being bound by theory some of such functions are described below:

It has been demonstrated that HBP's positively charged epitomes bind to negatively charged macromolecules such as the heparan sulphate and chondroitin sulphate side chains of the proteoglycans (Olofsson, A M. et al. 1999), which are present at the surface of nearly every adherent mammalian cells. Proteoglycans are proteins with long carbohydrate chains of the glucosaminoglycans (GAG) type attached. They have recently been recognized as an important part of the signaling mechanism between cells. The proteoglycans are today recognized as co-receptors that can influence how e.g. the growth factor interacts with its receptor. Co-receptors affect which signal molecules bind to the receptor, how strong the interaction is or how far the signal spreads. Co-receptors regulate such decisions as when the cell divides, what type of proteins it manufactures and even if it should die. HBP has been shown to bind to the carbohydrate part (e.g. heparan sulphate) of the syndecan family of proteoglycans, which play an important role in internalization of proteins. The binding of HBP to such proteoglycans lead to uptake of HBP into endothelial cells (Olofsson, A M et al., 1999) and probably other cell types as well. Heparan sulphate and similar highly charged negative molecules of the glucosaminoglycan type may therefore serve as binding sites for HBP, mediating many of its diverse regulatory functions. In this context it is should be noted that heparan sulphate and similar glucosaminoglycans are not just simple negatively charged molecules mediating a non-specific ionic interaction. In contrast e.g. the heparan sulphate are synthesized such that very diverse and subtle variations in the structure are achieved. Accordingly, the synthesized heparan sulphate molecules may fit only very specific positively charged epitopes, such as the ones found on the surface of HBP. The heparan sulphates and similar proteoglycans with GAG side chains may therefore be seen as a proper receptor or co-receptor for HBP.

HBP may also exploit its dipolar nature by activating the contact phase system. The contact phase system consists of HMWK and three other proteins which are closely bound together on the cell surface. HMWK is a large protein consisting of 6 domains, of which one (domain 4) contains the Bradykinin sequence. An electrostatic binding from a positively charged histidin-rich area in domain 5 of HMWK to negatively charged heparan sulphate (Renne, T. et al., 2000) and chondroitin sulphate (Renne, T. et al., 2001) proteoglycans contribute significantly to the binding of HMWK to cell surfaces. The activation of the contact phase system requires that the individual components (HMWK, fXII and pre-kallikrein) are brought in close contact to each other and probably also that certain conformational changes are induced. Heparin-binding protein (HBP) has been shown to play a pivotal role in activating the contact phase system (Gautam, N. 2001), and to be capable of highly effectively displacing HMWK from GAG in an in vitro model (Renne, T. 1999). This occurs most likely by formation of two electrostatic bindings, one between the negatively charged GAG on the cell surface and HBP's strongly positively charged surface area, and another between the positively charged domain 5 of HMWK and HBP's negatively charged surface area.

Further, in addition to the above-mentioned highly charged binding sites HBP also carries other putative binding sites, such as binding sites for the Lipid A part in LPS and for interaction with and activation of Protein Kinase C (PKC), see Iversen 1997 for a review.

Monofunctional HBP Peptides

The peptides according to the invention having agonistic or antagonistic properties to the putative binding sites for HBP are of considerable pharmaceutical interest as drug candidates for the prevention and/or the treatment of infections, local and systemic inflammatory disorders, asthma, systemic inflammatory response syndrome (SIRS), degenerative diseases (Alzheimer's disease), pain and other serious diseases and disorders (see table 1 in the Experimental section).

As outlined above HBP is by nature designed for initiation and regulation of local inflammatory defense to invading bacteria. In the situation of a local infection intact HBP is an ideal molecule for initiating, coordinating and regulating all the many different protecting mechanisms against the invading bacteria. In such situation a virulent inflammatory defense as initiated by HBP is fully appropriate and is probably needed to ensure survival of the individual. In agreement with this presumed beneficial effect of HBP, intact HBP has in animal studies been shown to be useful in prevention and treatment of severe life threatening infections and sepsis. However, in a therapeutic situation (by therapeutic is here to be understood both preventive and proper therapeutic interventions) HBP is administered differently from the natural way and therefore not all of its multiple effects may be needed or desirable.

Peptides with only one or some of the intact HBP molecule functions may therefore have significant advantages compared to the intact HBP for the treatment of specific conditions because they may be more specific, have different threshold for activation by a given process or be more powerful (displaying higher maximal efficacy).

Further, it is within the scope of the invention to provide peptides having a single function, i.e. mono-functional peptides, inhibiting specific HBP mediated processes. Below is a description of therapeutic applications, wherein the present peptides may be employed.

In the treatment of severe life threatening infections with HBP the monocyte activating and stimulating function may be the most important. To treat a lung infection for instance, HBP will most likely have to be administered systemically (e.g. as a subcutaneous injection or infusion). When administered at a site distant from the infection the ability of intact HBP to induce capillary leakage may not be advantageous, since this could lead to accumulation of neutrophils and edema formation at the administration site. While such potential side effect may be fully acceptable considering the ability of HBP to prevent or treat life-threatening infections, the use of a mono-functional HBP peptide according to the invention is to be preferred. The mono-functional peptide may have an increased ability to activate monocytes and a decreased ability to induce capillary leakage.

Monocytes play a significant role not only in eliminating bacteria but also in eliminating certain cancer cells. A mono-functional peptide with increased efficacy for stimulating the cytotoxic ability of the monocytes and macrophages would be highly desirable.

Many degenerative diseases (e.g. Alzheimer's disease) are characterized by an increased program cell death—i.e. and increased apoptosis. This means, that the cells die faster. Agents preventing or delaying apoptosis could conceivably be of use for slowing down the development of such degenerative diseases. A mono-functional peptide of the invention having a high anti-apoptosis efficacy, but no inflammatory potential may be a potential candidate for intervention in degenerative diseases.

Further, while the inflammatory defense initiated and regulated by intact HBP is needed to insure elimination of local bacterial infections it is not ideally designed to combat systemic infections, which spread to the whole body. In such circumstances, the inflammatory response may lead to damages to the organs and even to death of the organism if not controlled or stopped in time. Furthermore, the organism may react with an inflammatory response in situations where there is no infection. In such situations the inflammatory response is not only needless it is also highly damaging to the body. As an example, patients exposed to a trauma e.g. a car accident may develop a systemic inflammatory response caused by the extensive tissue damage. This may lead to hypotension, activation of the coagulation system, formation of clots and subsequent bleeding due to increased fibrinolysis, respiratory distress and failure of vital organs, such as the liver, kidney and the heart. The mechanisms leading to such Systemic Inflammatory Response Syndrome (SIRS) and Multiple Organ Failure (MOF) have not been fully elucidated. Without being bound by theory the interaction and contribution of HBP to the activation of the contact phase system probably plays a significant role.

According to the invention it is of significant interest to provide antagonist to HBP to use in the prevention and/or the treatment of such serious disorders. While antibodies to HBP may be highly useful in several clinical situations characterized by increased activation of the contact phase system and increased Bradykinin release, small peptide HBP antagonists may have several advantages. They may be used in a wider range of diseases and disorders due to their smaller size and presumed better tissue penetration.

In another aspect of the present invention a more effective means of preventing bradykinin mediated disease processes is provided. As mentioned above anti-HBP antibodies are obvious candidates, but small peptide HBP antagonists of the invention may have significant advantages. Bradykinin plays a role in the development of SIRS amongst other diseases. Bradykinin exerts its effect by interaction with specific receptors. Numerous bradykinin antagonists have been synthesized in the search for new drugs, which can prevent the action of bradykinin in conditions, such as circulatory and endotoxic shock, rhinitis and other allergic conditions, chronic inflammatory diseases such as rheumatoid arthritis, and colitis ulcerosa and brain edema. Although some of them have shown to be of some clinical use the effect has in general been less than expected considering the central mediator role of bradykinin. Without being bound by theory one reason might be that an antagonist only partly blocks the effect of bradykinin, but probably more likely that the antagonist in a therapeutic situation is often given after a significant amount of bradykinin has been released and has exerted its effects on the receptors.

In a further aspects of the present invention mono-functional, non-toxic agonists which bind LPS or other endotoxins, such as PGN, LTA or other cell wall components from bacteria with the same or higher affinity as Polymyxin B would have significant therapeutic potential for the treatment of sepsis. Endotoxins from both Gram negative (LPS) and Gram positive (PGN, LTA) bacteria play a significant role in the development of septic shock. It has recently been shown that removal of endotoxin (LPS) from the blood of septic patient by passage of the patient's blood through a Polymyxin B—column significantly reduces mortality. Polymyxin B binds LPS. Polymyxin B has certain structural similarities with HBP, which also binds LPS.

### Production

The peptides of the present invention may be prepared by conventional synthetic methods, recombinant DNA technologies, enzymatic cleavage of full-length proteins which the peptide sequences are derived from, or a combination of said methods.

Synthetic Preparation

The methods for synthetic production of peptides are well known in art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

Peptides may for example be synthesised by using Fmoc chemistry and with Acm-protected cysteins. After purification by reversed phase HPLC, peptides may be further processed to obtain for example cyclic or C- or N-terminal modified isoforms. The methods for cyclization and terminal modification are well-known in the art and described in detail in the above cited manuals.

The DNA sequence encoding a peptide or full-length protein of the invention may be prepared synthetically by established standard methods, e.g. the phosphoamidine method described by Beaucage and Caruthers, 1981, Tetrahedron Lett. 22:1859-1869, or the method described by Matthes et al., 1984, EMBO J. 3:801-805. According to the phosphoamidine method, oligonucleotides are synthesized, e.g. in an automatic DNA synthesizer, purified, annealed, ligated and cloned in suitable vectors.

Recombinant Preparation

The peptides of the invention may also be produced by use of recombinant DNA technologies. The DNA sequence encoding a peptide may be prepared by fragmentation of the DNA sequences encoding a full-length protein, which the peptide is derived from, using DNAase I according to a standard protocol (Sambrook et al., Molecular cloning: A Laboratory manual. 2 rd ed., CSHL Press, Cold Spring Harbor, N.Y., 1989). The present invention relates to the full-length protein being selected from the group of proteins comprising human HBP (SEQ ID NO:1), porcine HBP (SEQ ID NO: 588) and human neutrophil elastase (SEQ ID NO: 589), said proteins being encoded by the DNA sequences set forth in SEQ ID NO: 590, SEQ ID NO: 591 and SEQ ID NO: 592, correspondingly. The DNA encoding the full-length proteins may alternatively be fragmented using specific restriction endonucleases. The fragments of DNA are further purified using standard procedures described in Sambrook et al., Molecular cloning: A Laboratory manual. 2 rd ed., CSHL Press, Cold Spring Harbor, N.Y., 1989.

The DNA sequence encoding a full-length protein may also be of genomic or cDNA origin, for instance obtained by preparing a genomic or cDNA library and screening for DNA sequences coding for all or part of the full-length protein by hybridization using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primers, for instance as described in U.S. Pat. No. 4,683,202 or Saiki et al., 1988, Science 239:487-491.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding a peptide or a full-length protein should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the SV 40 promoter (Subramani et al., 1981, Mol. Cell Biol. 1:854-864), the MT-1 (metallothionein gene) promoter (Palmiter et al., 1983, Science 222: 809-814) or the adenovirus 2 major late promoter. A suitable promoter for use in insect cells is the polyhedrin promoter (Vasuvedan et al., 1992, FEBS Lett. 311:7-11). Suitable promoters for use in yeast host cells include promoters from yeast glycolytic genes (Hitzeman et al., 1980, J. Biol. Chem. 255:12073-12080; Alber and Kawasaki, 1982, J. Mol. Appl. Gen. 1: 419-434) or alcohol dehydrogenase genes (Young et al., 1982, in Genetic Engineering of Microorganisms for Chemicals, Hollaender et al, eds., Plenum Press, New York), or the TPI1 (U.S. Pat. No. 4,599,311) or ADH2-4c (Russell et al., 1983, Nature 304:652-654) promoters. Suitable promoters for use in filamentous fungus host cells are, for instance, the ADH3 promoter (McKnight et al., 1985, EMBO J. 4:2093-2099) or the tpiA promoter.

The coding DNA sequence may also be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) promoters. The vector may further comprise elements such as polyadenylation signals (e.g. from SV 40 or the adenovirus 5 Elb region), transcriptional enhancer sequences (e.g. the SV 40 enhancer) and translational enhancer sequences (e.g. the ones encoding adenovirus VA RNAs).

The recombinant expression vector may further comprise a DNA sequence enabling the vector to replicate in the host cell in question. An example of such a sequence (when the host cell is a mammalian cell) is the SV 40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hydromycin or methotrexate.

The procedures used to ligate the DNA sequences coding the peptides or full-length proteins, the promoter and the terminator, respectively, and to insert them into suitable vectors containing the information necessary for replication, are well known to persons skilled in the art (cf., for instance, Sambrook et al., op. cit.).

To obtain recombinant peptides of the invention the coding DNA sequences may be usefully fused with a second peptide coding sequence and a protease cleavage site coding sequence, giving a DNA construct encoding the fusion protein, wherein the protease cleavage site coding sequence positioned between the HBP fragment and second peptide coding DNA, inserted into a recombinant expression vector, and expressed in recombinant host cells. In one embodiment, said second peptide selected from, but not limited by the group comprising glutathion-S-reductase, calf thymosin, bacterial thioredoxin or human ubiquitin natural or synthetic variants, or peptides thereof. In another embodiment, a peptide sequence comprising a protease cleavage site may be the Factor Xa, with the amino acid sequence IEGR, enterokinase, with the amino acid sequence DDDDK, thrombin, with the amino acid sequence LVPR/GS, or Acharombacter lyticus, with the amino acid sequence XKX, cleavage site.

Host Cell

The host cell into which the expression vector is introduced may be any cell which is capable of expression of the peptides or full-length proteins, and is preferably a eukaryotic cell, such as invertebrate (insect) cells or vertebrate cells, e.g. Xenopus laevis oocytes or mammalian cells, in particular insect and mammalian cells. Examples of suitable mammalian cell lines are the HEK293 (ATCC CRL-1573), COS (ATCC CRL-1650), BHK (ATCC CRL-1632, ATCC CCL-10) or CHO (ATCC CCL-61) cell lines. Methods of transfecting mammalian cells and expressing DNA sequences introduced in the cells are described in e.g. Kaufman and Sharp, J. Mol. Biol. 159, 1982, pp. 601-621; Southern and Berg, 1982, J. Mol. Appl. Genet. 1:327-341; Loyter et al., 1982, Proc. Natl. Acad. Sci. USA 79: 422-426; Wigler et al., 1978, Cell 14:725; Corsaro and Pearson, 1981, in Somatic Cell Genetics 7, p. 603; Graham and van der Eb, 1973, Virol. 52:456; and Neumann et al., 1982, EMBO J. 1:841-845.

Alternatively, fungal cells (including yeast cells) may be used as host cells. Examples of suitable yeast cells include cells of Saccharomyces spp. or Schizosaccharomyces spp., in particular strains of Saccharomyces cerevisiae. Examples of other fungal cells are cells of filamentous fungi, e.g. Aspergillus spp. or Neurospora spp., in particular strains of Aspergillus oryzae or Aspergillus niger. The use of Aspergillus spp. for the expression of proteins is described in, e.g., EP 238 023.

Culture Medium

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be prepared according to published recipes (e.g. in catalogues of the American Type Culture Collection).

The peptides or full-length proteins recombinantly produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. HPLC, ion exchange chromatography, affinity chromatography, or the like.

### Experimentals

Identification of and Screening for Active HBP Peptide Sequences

Any HBP peptide sequence with 4 or more amino acid residues may be able to exercise an agonistic or antagonistic function against one or more of HBP putative binding sites or receptors (for simplicity just called HBP receptors below). However, certain sequences and surface areas may be identified as more interesting than others. Several measures to identify potentially interesting sequences with agonistic or antagonistic functions to HBP receptors were taken.

Firstly, the peptide sequence of HBP and proteins closely similar to HBP within the same species i.e. Homo sapiens were investigated. HBP is structurally very similar to human neutrophil elastase (hHNE), and specific HBP functions may be found in the areas of human HBP, which are non-identical to the sequences of hHNE, i.e. in the areas of the HBP molecule, which have not been conserved during evolution.

Secondly, it is hypothesized that such peptide sequences in HBP, which are conserved between species might be of particular interest.

Thirdly, it is hypothesized that sequences in other species than Homo sapiens e.g. the pig which are closely similar but not identical to the corresponding human sequence might have other abilities than the human sequence, e.g. be antagonistic instead of agonistic.

Finally, it is hypothesized that among the sequences identified as outlined above, the more interesting sequences may have to be found on the surface of the molecule. As the two more important known receptor-like surface areas (epitopes) are both highly charged it is hypothesized that sequences with high density of charged amino acids would be of particular interest.

Screening of HBP Peptide Sequences for Biological Activity

Among the many sequences identified by the above approach, only some will have desirable biological and pharmaceutical functions. The number of possible combinations (i.e. different amino acid sequences) is astronomical. As an example the theoretical number of different peptides consisting of 25 amino acids is 3.36*10³². Even though some sequences based on the above outlined considerations can be identified as more interesting than others, a high number of different peptides will have to be screened for interesting biological and pharmaceutical properties.

It Is therefore important to have highly reliable high capacity assay systems to identify HBP peptides and analogues hereof with pharmaceutical potential. In this contest it should be realized that testing of peptides to be useful for preventive or therapeutic purposes in humans, should be done in a human system. Intact HBP from one species does not necessarily react identical in other species and use of an animal test system e.g. a rat system for screening of the biological functions of an HBP derived peptide to be used in humans could easily be misleading. Below examples on screening assays to be used for identification of peptides with pharmaceutical potential for prevention and treatment of infectious diseases (e.g. pneumonia), severe inflammatory disorders (e.g. SIRS) and degenerative disorders (e.g. Alzheimer's disease) are presented.

### EXAMPLE 1

### Screening for Inflammatory and Anti-inflammatory Potential of HBP Derived Peptide.

Human whole blood (WB) contains besides red cells, platelets and plasma the white blood cells including the neutrophils and monocytes. Neutrophils and monocytes have receptors for bacterial products such as LPS, PGN and LTA. The bacterial products react directly or via specific binding proteins to receptors on the monocytes thereby stimulating them to secrete and release inflammatory cytokines comprising, but not limited to IL-1, IL-6, and TNF-α. HBP has in itself no measurable effect on cytokine secretion, but significantly amplifies cytokine synthesis and secretion induced by bacterial products. In the assay type described the amplification of 160 μmol HBP per ml WB in general leads to at least three-fold amplification of the cytokine secretion.

In the example to be described LPS from the E. coli is used to stimulate the monocytes in WB (anti-coagulated by use of citrate) and the activity is measured by subsequently quantifying IL-6 in plasma separated from WB. The activity of HBP, HBP derived peptides and analogues hereof are evaluated by their ability to:
1. increase IL-6 secretion in absence of bacterial products
2. increase IL-6 secretion in presence of bacterial products
3. decrease IL-6 secretion in presence of bacterial products
4. inhibit amplification of cytokine secretion induced by intact HBP

An ideal HBP peptide agonist will display the same ability as intact HBP itself in this system, i.e. it will have no activity itself when added to WB but when added simultaneously with LPS or another bacterial product it should stimulate the IL-6 secretion with a factor of at least 3, preferably 4 or more. If the peptide itself stimulates IL-6 secretion in options or of LPS or other bacterial products, it may lead to a systemic hyper-inflammation in organism which is not desirable.

Reagents and Methods

All operations must be carried out in LAF cabinet by observance of stringent aseptic techniques. All test tubes, pipette tips etc. must be pyrogen-free. Buffers must be prepared by use of sterile, pyrogen water, preferably water for injection. Use 0.1% pyrogen-free BSA/PBS for all dilutions.

Add 20 μl of HBP derived peptide (in concentrations from 25 to 2500 μM) to 100 μl freshly drawn (less than 4 hours old) citrate whole blood from a healthy human volunteer. Add 20 μl bacterial component (LPS, LTA or PGN) in concentrations from 5 to 5000 ng/ml, preferably 50 to 500 ng/ml. Mix well and incubate for 16-18 hours in an atmosphere of 5% carbon dioxide and at least 95% relative humidity. At the end of the incubation add at least 5 volumes (700 μl) 0.1% BSA/PBS. Mix well. Centrifuge 10 min. at 10.000 g. Aspirate 500 μl supernatant. Determine the level of IL-6 by a specific human immune assay for human IL-6 with sensitivity of at least 3 pg/ml, e.g. Human IL-6 Kit from RnD Systems (cat. no. D 6050).

Negative controls: 100 μl WB plus 40 μl 0.1% BSA/PBS. Positive control: 100 μl WB plus 20 μl LPS (same concentration as used for testing the peptide) and 20 μl 0.1% BSA/PBS.

### EXAMPLE 2

### Screening for Anti-Apoptotic Potential of HBP Derived Peptides and Analogues

The screening for anti-apoptotic peptides is carried out essentially as described by Shrotri M S. et al., 2000.

Isolation of Human Neutrophils (PMNs)

Peripheral blood from normal volunteers was collected and PMNs were separated by density gradient using Ficoll-Hypaque (Sigma Chemical Co.). PMNs obtained were divided into 1-ml samples with 3.0 3 106 cells/ml and were treated and analyzed as per the protocols described below.

Cell Fixation and Staining Protocol for Analysis of Apoptosis

Cell pellets were obtained by centrifugation at 300 g and fixed with 1% paraformaldehyde for 15 min at 4° C. Cells were washed twice and permeabilized with 70% ice-cold ethanol and stored at 220° C. Cells samples were washed twice and stained by the terminal dUTP nick-end labeling (TUNEL) assay using the APO-BRDU kit (Phoenix Flow Systems, San Diego, Calif.), following the manufacturer's instructions. Briefly, in this assay, the enzyme terminal deoxynucleotidyl transferase (TdT) catalyzes the addition of nucleotides to DNA strand breaks in the apoptotic cells, which are subsequently labeled with fluorescein isothiocyanate (FITC)-conjugated antinucleotide antibodies. The fluorescent cells are the apoptotic cells that are then identified or assessed using the flow cytometer (EPICS Elite, Beckman-Coulter, Hialeah, Fla.). These apoptotic cells were also visually confirmed by confocal microscopy (Meridian Instruments Inc., Okemos, Mich.).

Time zero group. Certain samples were fixed immediately after isolation, permeabilized, and stored at 220° C. for TUNEL assay, as described earlier. These samples were designated as time zero, in which no culturing was involved.

24-h culture groups. Certain samples were re-suspended after isolation in 10% fetal bovine serum (serum-enriched group) with 25 μg of control protein (BSA) or 25 μg of HBP per ml. sample. Samples were incubated in 0% serum or RPMI (serum-deprived group) with control protein, HBP or HBP derived peptide. After 24 h culture in a humidified CO2 incubator, cells were fixed, permeabilized, and stored at 220° C. for later TUNEL assay, as described above.

Identification of Anti-Apoptotic Peptides

Addition of 25 μg HBP per ml sample typically decreases apoptosis from about 70% to 45%. Peptides decreasing apoptosis to the same or greater extent when used in equimolar amounts should be considered anti-apoptotic.

### EXAMPLE 3

### Identification of a Highly Potent Anti-Inflammatory HBP Peptide

As an example on the use of the methods outlined above to predict an HBP peptide sequence it is disclosed how a highly anti-inflammatory novel HBP peptide sequence is identified.

The innate immune response is activated by pattern recognition receptors (toll like receptors) on monocytes, neutrophils and other immune cells. HBP increase the sensitivity of these pattern recognition receptors for their response to the specific pattern motifs on the cell wall of both gram negative and gram positive bacteria as well as on fungi and other infective agents. Serine proteases and more important serine proteases with mutations in the catalytic site, but with a highly conserved serine protease fold such as HBP, play a pivotal role in innate immunity. Invertebrates have only innate immune response as the specific immune system was first developed with the evolution of the bonefish. Several serine proteases with mutations in the active site has been studied in a number of invertebrates and the Trichoplusia ni larval has an HBP-like serine protease. The hallmark in the mutation in the active site in the HBP from man, pig and Trichoplusia ni is an Histidin (H) to Serine (S) mutation, a conserved Aspartic acid (D) whereas the Serine (S) mutation is random. The active site with these mutations is therefore highly likely involved in the mechanism of action of the HBP family.

### Method

In the present context the term “h20-44” covers the human heparin binding protein sequence of amino acids numbers 20-44. Further, by the term “p20-44” is meant the porcine heparin binding protein sequence of amino acids numbers 20-44.

The human (h20-44) and porcine (p20-44) peptides were synthesized and tested in the screening assay described in Example 1. The following parameters were examined: (a) abililty to induce inflammation on their own (i.e. in the absence of any bacterial component), (b) ability to amplify or inhibit inflammation induced by a bacterial component, such as LPS from the Gram negative bacteria E. coli and PGN or Purified Cell Wall (PCW) from a Gram positive bacteria. As measure for immune stimulation secretion of the release of IL-6 in citrated whole blood was used. The peptides were tested in the concentrations 0.09, 0.18, 0.36 and 0.71 mg/ml blood. LPS was used at the concentration 100 ng/ml blood and PGN and PCW were used at 50 μg/ml blood.

### Results

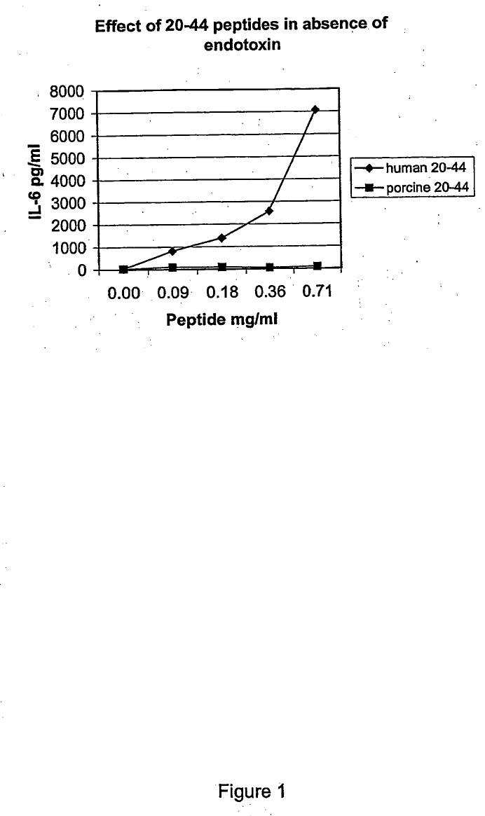

Human HBP 20-44 peptide induced a significant and dose-dependent increase in IL-6 secretion, whereas porcine HBP 20-44 displayed no significant or dose-dependent effect (see FIG. 1).

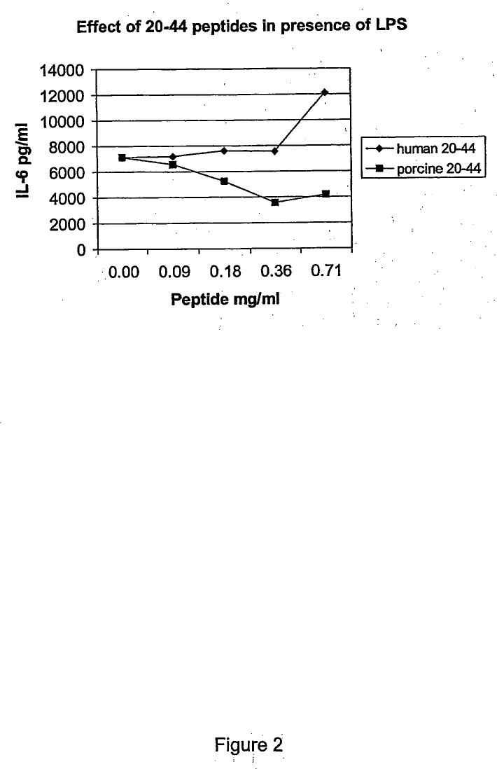

In blood stimulated with 100 ng/ml LPS, human HBP 20-44 did not significantly increase the IL-6 secretion up to 0.36 mg/ml peptide. At 0.71 mg/ml, human HBP 20-44 increased IL-6 secretion significantly, but the effect of human HBP 20-44 and LPS together was slightly less (12,130 pg/ml) than the sum of the IL-6 secretion induced by LPS and human HBP 20-44 individually (14,178 pg/ml) (see FIG. 2). In contrast porcine HBP 20-44 peptide significantly and dose-dependently inhibited the LPS induced IL-6 secretion.

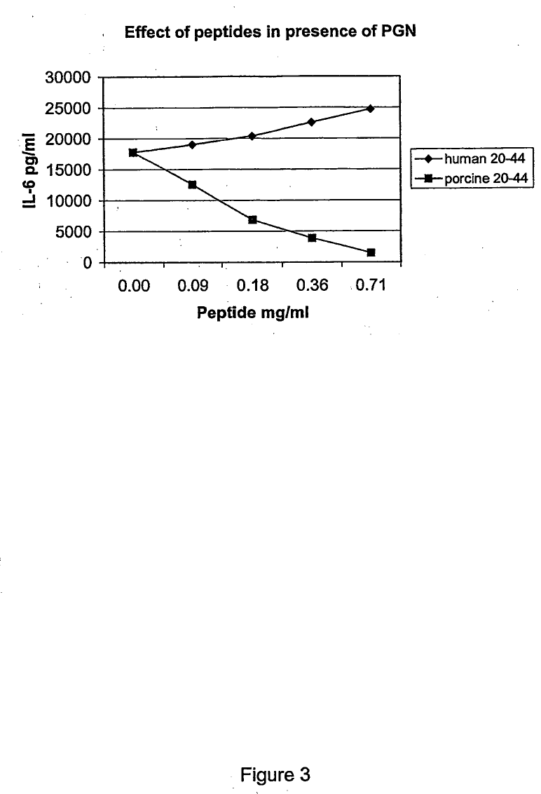

In blood stimulated with PGN (50 μg/ml) from Staphylococcus aureus human HBP 20-44 increased IL-6 secretion in dose-dependent way (see FIG. 3). In contrast porcine HBP 20-44 peptide significantly and dose-dependently inhibited the PGN induced IL-6 secretion.

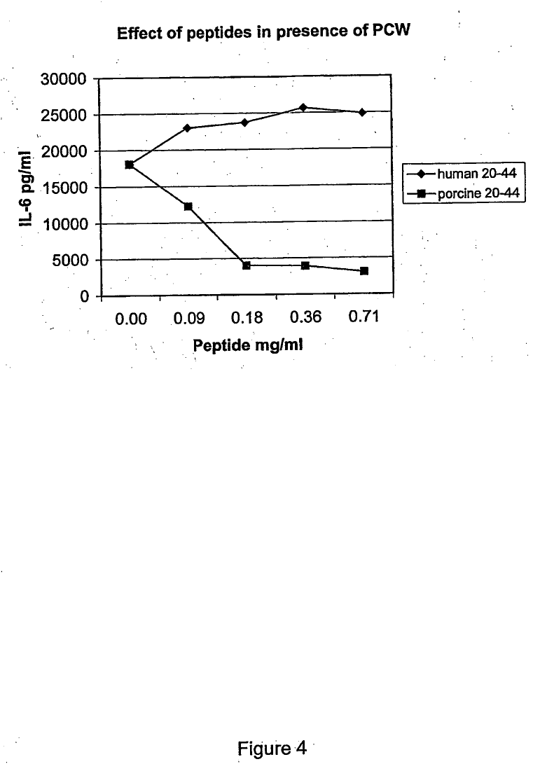

In blood stimulated with PCW (50 μg/ml) from Staphylococcus aureus human HBP 20-44 increased IL-6 secretion in dose-dependent way (FIG. 4). In contrast porcine HBP 20-44 peptide significantly and dose-dependently inhibited the PCW induced IL-6 secretion.

Conclusion

Human HBP 20-44 peptide surprisingly by itself significant stimulate secretion of the pro-inflammatory cytokine IL-6. In the presence of bacterial components human HBP 20-44 to some extent further increases the immune stimulation induced by the bacterial products. Human HBP 20-44 has previously been thought to act via its ability to bind to LPS and was presumed to be an LPS neutralizing agent. An LPS neutralizing agent would inhibit the immune response, but here it is shown that human HBP 20-44 is instead a powerful immune stimulating agent. In contrast the structurally very similar porcine HBP 20-44 peptide, which only deviates from its human counterpart by 7 amino acid substitutions was found to be a highly potent anti-inflammatory agent, which significantly decreases inflammation induced by bacterial components from both Gram negative and Gram positive bacteria. Porcine HBP 20-44 peptide thus holds significant potential for becoming a broadly applicable anti-inflammatory agents with indications ranging form treatment of chronic inflammatory diseases over re-perfusion injuries in myocardial and brain insults to the life-threatening systemic inflammatory response syndrome.

### EXAMPLE 4

### Modification of the C-Terminal and N-Terminal Amino Acid Residues in hHBP and pHBP

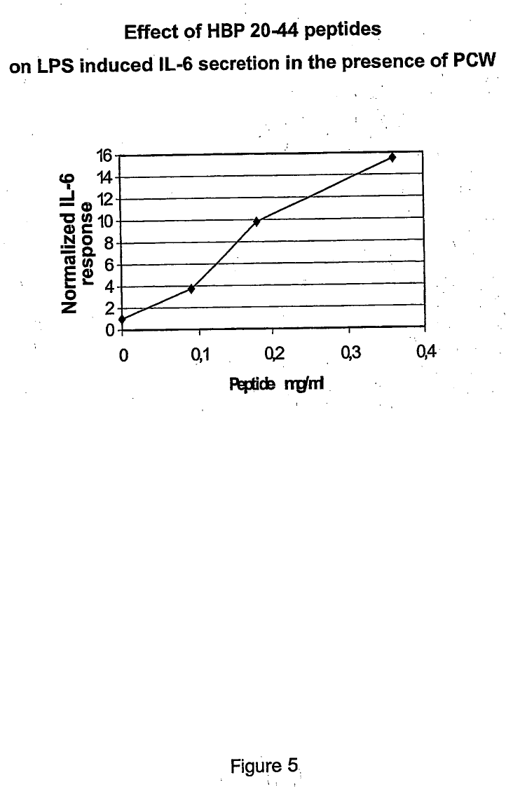

In human blood stimulated with 250 μg/ml PGN from S. aureus, hHBP 20-44 acetylated at the N-terminal amino group and amidated at the C-terminal carboxyl group stimulated the IL-6 secretion in a dose dependant way (FIG. 5). In FIG. 5 the IL-6 response has been normalized to the effect of PGN alone. Compared to FIG. 2 it appears that there is a more than ten fold stimulation of the PGN mediated IL-6 production for the N- and C-terminal substituted peptide.

### EXAMPLE 5

### Essential Amino Acid Residues in hHBP and pHBP Involved in Regulation of IL-6 Production

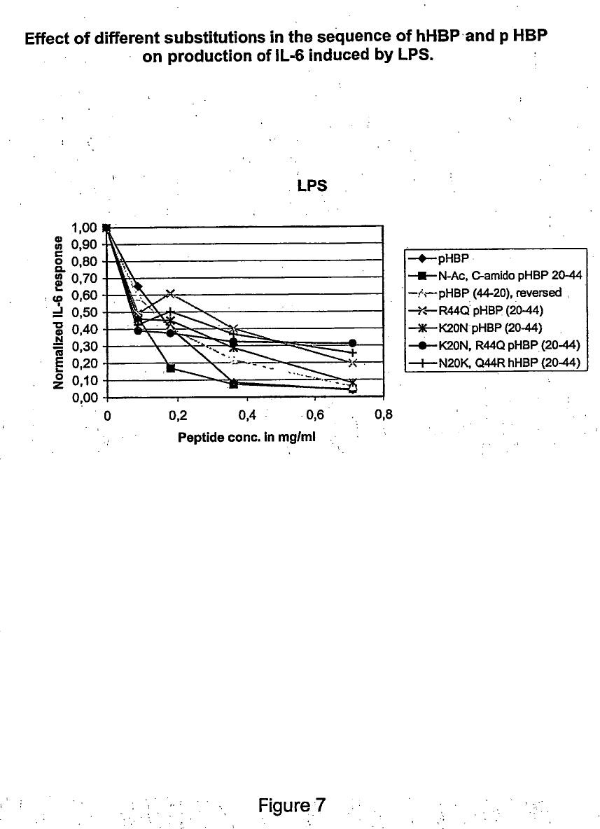

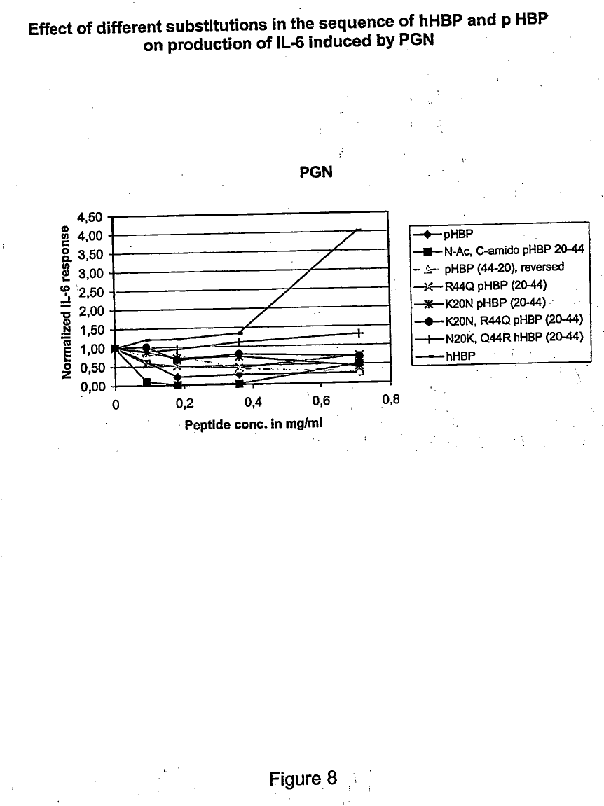

From FIGS. 7 and 8 it can be seen that when pHBP 20-44 was acetylated and amidated at the N- and C-terminal amino acids, respectively, the variant peptide became more inhibitory to the LPS and PGN induced IL-6 production compared to the unsubstituted peptide.

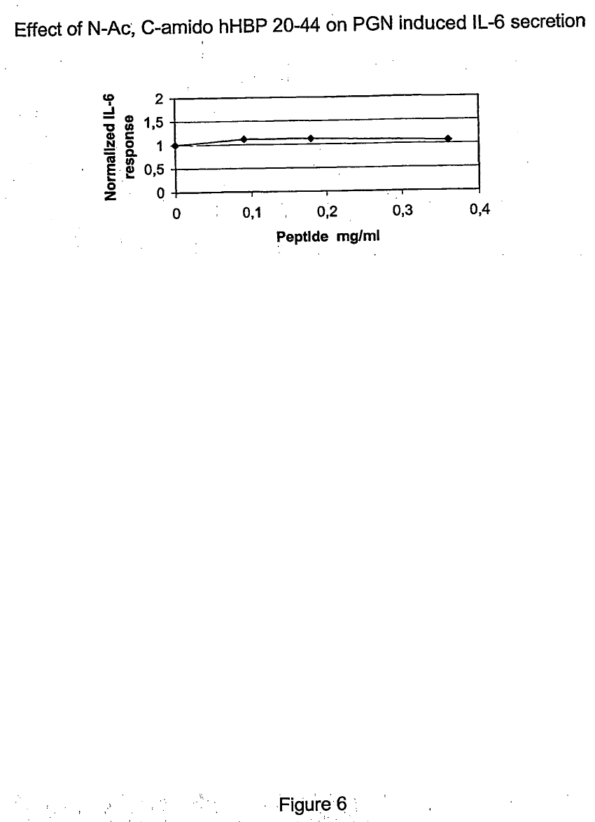

In order to identify essential amino acid residues in human and porcine HBP 20-44 involved in the effects observed (see FIGS. 1-4), several variants with amino acid substitutions in defined positions were tested. From FIG. 6 it appears that the substitution of arginine to glutamine in position 34 of pHBP 20-44 completely abolished the inhibitory effect of the peptide in the presence of PGN.

From FIGS. 7 and 8 it appears that when the N-terminal amino acid residue of the pHBP 20-44 was substituted with the human N-terminal amino acid residue, the variant became less inhibitory compared to the original porcine peptide in the presence of both LPS and PGN. When both the N- and C-terminal amino acid residues of hHBP 20-44 was substituted with the corresponding porcine amino acid residues, the variant became nearly non-stimulating compared to the unsubstituted human peptide in the presence of LPS and in the presence of PGN the substituted peptide became inhibitory to the IL-6 production. From FIGS. 7 and 8 it also appears that the same pattern was observed when the C-terminal amino acid residue in hHBP 20-44 was replaced with the porcine C-terminal amino acid residue, or when C-terminal amino acid residue in pHBP 20-44 was replaced with the human C-terminal amino acid residue.

TABLE 1
Potential applications for mono-functional peptides

| HBP | Mono-functional | Mono-functional |
|---|---|---|
| function | agonist | antagonist |
| Capillary leakage | | Edema, SIRS |
| Chemotaxis | Wound healing, | Re-perfusion injury, |
| | local infections | bran edema |
| Monocyte activation | Local and systemic | Inflammatory diseases, |
| | infections, (tumors) | SIRS |
| Activation contact | (Infection) | SIRS, post-operative |
| phase system | | bleeding, pain, |
| | | (inflammation) |
| Anti-apoptosis | Degenerative diseases | (Tumors) |
| Endotoxin binding | Endotoxin removal | |
| | from septic patients | |

1. A peptide having a sequence of at most 44 amino acid residues comprising a motif of the formula
X¹-X²-Cys-X³-X⁴-X⁵-X⁶-X⁷-X⁸-X⁹-X¹⁰-X¹¹-X¹²-X¹³-X¹⁴-X¹⁵-X¹⁶-X¹⁷-Cys-X¹⁸-X¹⁹,
wherein
X can be an amino acid sequence or a single amino acid residue selected either from Group 1 consisting of Ala, Gly, and Ser,
Group 2 consisting of Arg and Lys,
Group 3 consisting of His, Ile, Leu, Met, Phe, Pro, Thr, Val, Trp, and Tyr,
Group 4 consisting of Asn and Gln, or
Group 5 consisting of Ala, Asn, Arg, Gln, Gly, His, Ile, Leu, Lys, Met, Phe, Pro, Ser, Thr, Trp, Tyr, Val,
wherein
X¹ can be represented by a sequence consisting of 2-5 amino acid residues or an amino acid residue selected from Group 2;
X² is selected from Group 5 or Group 3;
X³, X¹⁵ and X⁴ are selected from Group 1;
X⁵ is Thr or selected from from Group 1;
X⁶, X¹¹, X¹², X¹³ and X⁷ are selected from Group 3;
X⁸ and X¹⁷ are selected from Group 1, 3 or 4;
X⁹ is selected from from Group 5, 1 or 3;
X¹⁰ is selected from from Group 2, 3 or 4;
X¹⁴ is Ser or selected from from Group 3;
X16 and X¹⁸ is selected from Group 1 or 3
X¹⁹ can be represented by a sequence consisting of 2-5 amino acid residues or a single amino acid residue selected from Group 5, 2, or 4,
with the proviso, that when X¹ includes Pro, then X¹⁹ is Gln.

2.-26. (canceled)

27. The peptide according to claim 1, wherein said peptide has the sequence KQGRHFCGGALIHARFVMTAASCFR (SEQ ID NO: 595).

28. The peptide according to claim 1, wherein said peptide has the sequence KQGRPFCGGALIHARFVMTAASCFR (SEQ ID NO: 596).

29. The peptide according to claim 1, wherein said peptide has the sequence KQGRHFCGGALIHPRFVMTAASCFR (SEQ ID NO: 597).

30. The peptide according to claim 1, wherein said peptide has the sequence KQGRPFCGGALIHPRFVMTAASCFR (SEQ ID NO: 598).

31. The peptide according to claim 1, wherein said peptide has the sequence RFCSAATLVFRPHVLAGACFPRGQK (SEQ ID NO: 599).

32. The peptide according to claim 1, wherein said peptide has the sequence NQGRPFCAGALVHPRFVLTAASCFR (SEQ ID NO: 600).

33. The peptide according to claim 1, wherein said peptide has the sequence KQGRPFCAGALVHPRFVLTAASCFQ (SEQ ID NO: 601).

34. The peptide according to claim 1, wherein said peptide has the sequence NQGRPFCAGALVHPRFVLTAASCFQ (SEQ ID NO: 602).

35. The peptide according to claim 1, wherein said peptide has the sequence KQGRPFCAGALVHPQFVLTAASCFR (SEQ ID NO: 603).

36. The peptide according to claim 1, wherein said peptide has the sequence LRGGHFCGATLIAPNFVMSAAHCVA (SEQ ID NO: 604).

37. The peptide according to claim 1, wherein said peptide has the sequence RRGGHFCGATLIARNFVMSAVHCVN (SEQ ID NO: 605).

38. The peptide according to claim 1, wherein said peptide has the sequence RSREYRCGGTLVSQRYILTAASCAA (SEQ ID NO: 606).

39. The peptide according to claim 1, wherein said peptide has the sequence NQGRHFCGGALIHARFVMTAASCFQ (SEQ ID NO: 594).

40. The peptide according to claim 1, wherein said peptide has the sequence KQGRPFCAGALVHPRFVLTAASCFR (SEQ ID NO: 593).

41.-52. (canceled)

53. The peptide according to claim 1, wherein said peptide is capable of inhibiting the secretion of cytokine IL-6 from monocytes.

54. The peptide according to claim 1, wherein said peptide is capable of stimulating the secretion of cytokine IL-6 from monocytes.

55.-56. (canceled)

57. A method of treatment of gram-negative bacterial infection comprising administering to an individual in need one or more peptides as defined in claim 1.

58. A method of treatment of gram-positive bacterial infection comprising administering to an individual in need one or more peptides as defined in claim 1.

59.-72. (canceled)

73. A pharmaceutical composition comprising a peptide as defined in claim 1.
